(19) United States
(12) Patent Application Publication
(10) Pub. No.: US 2025/0090581 A1
(43) Pub. Date: Mar. 20, 2025

(54) MULTISTEP PROCESS FOR CULTURING TUMOR-INFILTRATING LYMPHOCYTES FOR THERAPEUTIC USE

(71) Applicant: CBIO A/S, Søborg (DK)

(72) Inventors: Nikolaj Kirketerp-Møller, Copenhagen N (DK); Christina Friese, Vanløse (DK); Christina Heeke, Copenhagen NV (DK); Ulrik Cordes, Charlottenlund (DK)

(21) Appl. No.: 18/569,874

(22) PCT Filed: Jun. 17, 2022
(86) PCT No.: PCT/EP2022/066633

§ 371 Date: Dec. 13, 2023

(30) Foreign Application Priority Data
Jun. 17, 2021 (EP) 21180126.1

(51) Int. Cl. A61K 35/17; A61K 39/00; C12N 5/00; C12N 5/0783

(52) U.S. Cl. CPC A61K 35/17; A61K 40/11; C12N 5/0018; C12N 5/0636; C12N 2501/2302; C12N 2501/515; C12N 2501/599

(57) ABSTRACT

The present invention is targeted towards depleting suppressive cells, including regulatory T cells, and/or reinvigorating exhausted Tumor Infiltrating Lymphocytes (TILs) in vitro by co-culturing excised TIL containing tumor fragments (or tumor digest) with Tumor Microenvironment (TME) Stimulators, such as Immune Checkpoint Inhibitors (ICIs), Cytokines/interleukins, and/or inhibiting the effect of regulatory T cells secreted factors (such as inhibiting IL-10) thereby creating a favorable tumor microenvironment where inhibitory T-cells and/or signals are removed so that exhausted T-cells can expand faster, to higher numbers, and are more potent than currently established TIL expansion protocols. A time lapse of the use of TME stimulators is of interest.

### FIELD

The present invention is targeted towards depleting suppressive cells, including regulatory T cells, and/or reinvigorating exhausted tumor infiltrating lymphocytes (TILs) in vitro by co-culturing excised TIL-containing tumor fragments (or tumor digest) with Tumor Microenvironment (TME) Stimulators, such as Immune Checkpoint Inhibitors (ICIs), Cytokines/interleukins, and/or inhibiting the effect of factors secreted by suppressive cells, including regulatory T cells (such as IL-10) thereby creating a favorable tumor microenvironment where inhibitory T cells and/or signals are removed so that exhausted T cells can expand faster, to higher numbers, and are more potent than currently established TIL expansion protocols. A time lapse of the use of TME stimulators is of interest.

### BACKGROUND

Tumor infiltrating lymphocytes are associated with improved prognosis and progression-free survival in cancer patients undergoing immunotherapy such as the use of immune checkpoint inhibitors (ICIs) against CTLA-4 and PD-1/PD-L1.

However, still only a fraction of patients has a durable long-term response to such therapies as many other factors seem to be involved in the tumor microenvironment in the down regulation of the immune response. One of the key factors seems to be exhaustion of T cells resulting in the physical elimination and/or dysfunction of antigen-specific T cells. Factors involved in this exhaustion phenomenon involve surface markers expressed on tumor cells, lymphoid and mononuclear cells and soluble molecules secreted from regulatory T cells and NK cells in the tumor microenvironment (TME). But, also the lack of stimulatory factors such as interferon gamma and IL-2 is evident in the TME.

Reversal of T-cell exhaustion is a key target in the development of new classes of ICIs either as a monotherapy or in combination with already established therapies. However, as these targets often are also responsible for inducing immune tolerance avoiding autoimmune responses, systemic administration of inhibitors can cause serious side effects. In addition, administering T-cell stimulatory molecules such as IL-2 can also cause serious and sometimes fatal side effects and therefore needs to be managed by skilled clinicians. Some approaches have been taken to administer drug candidates locally into the tumor thereby possibly avoiding systemic side effects. However, as cancer cells are distributed all over the body in many metastatic patients, the likelihood of this approach to be successful under such circumstances can be questioned.

The use of tumor infiltrating lymphocyte (TIL) therapy has shown significant clinical benefit with objective response rates of up to 55% and complete responses in up to 20% of patients in various malignancies such as metastatic melanoma, head and neck and cervical cancer. In short, this kind of therapy leverages the in vitro expansion of autologous T lymphocytes by stimulating fragments from the excised tumor with IL-2, anti-CD3 antibodies and feeder cells and thereby growing these cells to billions before re-administering the T cells in back to the lymphodepleted patient followed by IL-2 infusion where after regression of the tumor is promoted.

TIL therapy is costly and takes time. It would therefore be advantageous to optimize the current methods and identify ways to shorten the duration for expansion of the TILs, increase the expansion rate, and also achieve more favorable phenotypes and functionality.

### SUMMARY

The present invention relates to a method for promoting regression of a cancer in a mammal by expanding tumor infiltrating lymphocytes (TILs) into a therapeutic population of TILs comprising: (a) culturing autologous T cells by obtaining a first population of TILs from a tumor resected from a mammal, (b) performing a depletion of suppressive cells, including regulatory T cells, and/or blocking negative signals by the addition of TME stimulators from the group of “Inhibitors” with or without cytokines (c) performing a first expansion by culturing the depleted population of TILs in a cell culture medium comprising one or more of the “cytokine” group and/or one or more of the substances from the “Stimulator” group to produce a second population of TILs; (d) performing a second expansion by supplementing the cell culture medium of the second population of TILs with additional IL-2 and/or other cytokines from the “cytokine” group, anti-CD3 antibodies, and antigen presenting cells (APCs), to produce a third population of TILs, wherein the third population of TILs is a therapeutic population; and (e) after administering nonmyeloablative lymphodepleting chemotherapy, administering to the mammal the therapeutic population of T cells, whereupon the regression of the cancer in the mammal is promoted.

### DETAILED DESCRIPTION

The present invention is targeted towards depleting suppressive cells, including regulatory T cells, and/or reinvigorating exhausted tumor infiltrating lymphocytes (TILs) in vitro by co-culturing excised TIL-containing tumor fragments with for example checkpoint inhibitors, stimulating the TILs with other cytokines/interleukins known to revert T-cell exhaustion, and/or inhibiting the effect of factors secreted by regulatory T cells (such as IL-10) thereby creating a favorable TME where exhausted T cells can expand faster, to higher numbers, and are more potent than currently established TIL expansion protocols.

This approach is possibly advantageous to systemically administered therapies as the in-vitro stimulation can be performed using dose levels that are much higher than would be tolerated in vivo. As an example, current TIL protocols utilize IL-2 at a concentration at 3-6,000 IU per mL, which is 5-10 times higher than the systemically recommended dose.

In addition, as T cells that have a higher affinity to tumor antigens might have an increased tendency to get exhausted, targeted in-vitro reinvigoration might help expanding T-cell clones with higher affinity that more aggressively can target the tumor thereby eventually leading to an improved clinical outcome of this novel approach to TIL therapy.

The present inventors have found that a depletion of suppressive cells, including regulatory T cells, and/or blocking negative signals by the addition of one or more TME stimulators from the group of “Inhibitors” to obtain a depleted population of TILs followed by performing a first expansion by culturing the depleted population of TILs in a cell culture medium comprising: one or more TME stimulators from the group of “cytokines”, and/or one or more of the TME stimulators from the “Stimulator” group to produce a second population of TILs leads to:
Higher proportion/total cell number of favorable TILs (CD8+, T cells with an effector memory phenotype etc.),
Faster growth rate,
Higher reactivity of the cultured T-cells (measured by “potency assay”),
Higher total number of reactive T-cells,
Higher number of tumor specificities,
Higher frequency of tumor-specific T cells, and/or
Higher T-cell repertoire (based on TCR sequencing).

Thus, the present invention relates to expanded tumor infiltrating lymphocytes (TILs) for use in treating a subject with cancer, the treatment comprising the steps of: a) culturing autologous T cells by obtaining a first population of TILs from a tumor resected from a mammal, b) performing a depletion of suppressive cells, including regulatory T cells, and/or blocking negative signals by the addition of one or more TME stimulators from the group of “Inhibitors” to obtain a depleted population of TILs with or without the addition of “cytokines”, c) performing a first expansion by culturing the depleted population of TILs in a cell culture medium comprising: one or more TME stimulators from the group of “cytokines”, and/or one or more of the TME stimulators from the “Stimulator” group to produce a second population of TILs, d) performing a second expansion by supplementing the cell culture medium of the second population of TILs with additional IL-2 and/or other cytokines from the “cytokine” group, anti-CD3 antibody, and antigen presenting cells (APCs), to produce a third population of TILs, wherein the third population of TILs is a therapeutic population; and e) after administering nonmyeloablative lymphodepleting chemotherapy, administering to the mammal the therapeutic population of T cells, wherein the T cells administered to the mammal, optionally followed by IL-2 infusion, whereupon the regression of the cancer in the mammal is promoted.

By “tumor infiltrating lymphocytes” or “TILs” herein is meant a population of cells originally obtained as lymphocytes that have left the bloodstream of a subject and migrated into a tumor. TILs include, but are not limited to, CD8+ cytotoxic T cells (lymphocytes), Th1 and Th17 CD4+ T cells (CD4+ helper cells), natural killer cells, dendritic cells and MI macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein. TILs can generally be defined either biochemically, using cell surface markers, or functionally, by their ability to infiltrate tumors and induce tumor cell killing. TILs can be generally categorized by expressing one or more of the following biomarkers: CD4, CD8, TCR ab, CD27, CD28, CD56, CCR7, CD45Ra, CD95, PD-1, LAG-3, TIM-3, CD69, CD103, CD107a, TNFa, IFNg, CD3, and CD25. Additionally, and alternatively, TILs can be functionally defined by their ability to infiltrate solid tumors upon reintroduction into a patient. TILs may further be characterized by potency—for example, TILs may be considered potent if, for example, interferon (IFN) release is greater than about 50 μg/mL, greater than about 100 μg/mL, greater than about 150 μg/mL, or greater than about 200 μg/mL or interferon (IFN), tumor-necrosis-factor (TNF) production can be detected intracellularly and CD107a on the cell surface upon stimulation with coated beads or tumor cells (tumor cell lines or tumor digest). Functionality of these stimulated TILs can for example be further characterized by classification into single-, double-, or triple positivity for TNF and/or IFN and/or CD107a, whereby triple positive TILs are considered the most functional. Potency of the TIL product can be further characterized by analyzing direct tumor cell killing by detection of apoptosis and/or proliferation of tumor cells.

Number of specificities of the CD8+ T cells and their frequency in the TIL product can be defined by staining TILs with MHC multimer complexes displaying tumor peptides of interest that can be recognized by CD8+ T cells and/or by sequencing the T cell receptor repertoire.

The present inventors have found a higher percentage of CD8 T cells expressing markers associated with tumor-specificity (exhaustion markers), when performing the methods of the present invention, especially in JAB TD (time delay—see for example FIG. 7 and FIG. 9). An increase in % CD8 cells with TME stimulators has been repeatedly associated with a better response to ACT. Thus, a time delay (TD) can yield a higher percentage of CD8 T cells expressing markers associated with tumor-specificity (exhaustion markers), as shown in e.g. FIG. 7 and FIG. 9. An embodiment of the present invention relates to a method for expanding tumor infiltrating lymphocytes (TILs) into a therapeutic population, and the medical uses of this therapeutic population, according to the methods and uses described here, wherein in step c) the one or more TME stimulators added given in time delay (as described herein). This leads to a higher percentage of CD8 T cells expressing markers associated with tumor-specificity (exhaustion markers). A preferred embodiment relates to the use of JAB in combination for steps b) and c), as described herein. A and B can be added in step b) while J is added in step c). An embodiment of the present invention relates to a product comprising a clinically relevant number of TILs with a higher percentage of CD8 T cells expressing markers associated with tumor-specificity (exhaustion markers). This can be measured by either pMHC tetramer/multimer staining as number and frequencies of cancer-specific populations (tetramer staining as done in FIGS. 17-19) or by t cell receptor sequencing. These methods can be used to confirm that the product has a higher percentage of CD8 T cells expressing markers associated with tumor-specificity. The present data shows for example that JAB/JAB TD samples have >1 population whereas the two IL-2 samples have 0 or 1.

The present inventors have found increased viable CD3 and CD8 cells with TME stimulators, as can be seen in FIG. 8. The latter has been repeatedly associated with a better response to adoptive cell therapy (ACT). The culture conditions result in similar numbers of CD4 T cells compared to traditional IL2 stimulation. Thus, when step b) and step c) are performed in time lapse, increased viable CD3 and CD8 cells are produced in the methods of the present invention. A preferred embodiment relates to the use of JAB in combination for steps b) and c), as described herein. A and B can be added in step b) while J is added in step c).

FIG. 9 shows CD8 T cells with higher expression of particular activation markers, also called exhaustion markers (shown in A-C), have been associated to tumor-specific cells. Although similar % of cells expressing LAG3 was observed, there was a tendency of higher % TIM3 with TME stimulators compared to IL2 and increased % BTLA+ cells in JAB and JAB TD conditions, which may indicate an increase in % tumor-specific cells. BTLA expressing cells were associated with better outcome of ACT. Of note, JAB TD conditions tend to have higher % BTLA+ cells. Thus, these effects are seen when step b) and step c) are performed in time lapse. A and B can be added in step b) while J is added in step c).

FIG. 10 shows more Tem cells when TME stimulators are used (see especially with JAB and JAB+C+D TD conditions). The Tem subset has been repeatedly associated with a favorable outcome to ACT. The combination of JAB, and especially A and B in step b) and J in step c) is therefore favourable.

(Krishna, S et al., Stem-like CD8 T cells mediate response of adoptive cell immunotherapy against human cancer, Science 370, 1328-1334 (2020)). showed that responders to ACT had higher numbers of stem-like CD39-CD69-CD8 T cells in the infusion products, whereas non-responders tended to have more CD39+ CD69+ CD8 T cells. With TME stimulators, especially in time delay (TD) conditions the present inventors are able to increase the stem-like CD8 T cells and decrease the CD39+ CD69+ cells. Increased % of stem-like CD39-CD69-CD8 T cells with TME stimulators was also observed. Increased number of these stem-like CD8 T cells particularly in JAB TD condition (It was shown that responders to ACT had higher numbers of stem-like CD39− CD69−CD8 T cells in the infusion products). Thus, these effects are seen when step b) and step c) are performed in time lapse. A and B can be added in step b) while J is added in step c) to give the effect.

The time delay results in the examples of the present disclosure shows favorable results with more T and CD8 T cells, increased % CD28+ CD8 T cells, less % CD39+ CD69+ and more % CD39-CD69-CD8 T cells.

The examples show increased number of reactive CD8 T cells with TME stimulators. Also, increased number of triple positive CD8 T cells with TME stimulators (higher cytotoxic—tumor killing—capacity).

FIG. 12 shows more % of CD28+ CD8 T cells with TME stimulators, and especially in JAB TD conditions, and tendency of higher % CD28+ in TD conditions in general (indication of more cells capable of responding to further stimulation).

FIG. 13 shows cells triple positive for reactivity markers, that might show a higher expression of costimulatory and effector molecules, increasing their cytotoxic capacity. Higher total numbers of reactive, specifically triple+ T cells might be beneficial for ACT outcome. CD8 T cells cultures with the TD tend to have higher percentage of reactive and triple+ cells, as well as higher total numbers.

FIG. 14 shows cells triple positive for reactivity markers might show a higher expression of costimulatory and effector molecules, increasing their cytotoxic capacity. Higher total numbers of reactive, specifically triple+ T cells might be beneficial for ACT outcome. CD8 T cells cultured with the TD tend to have higher percentage of reactive and triple+ cells, as well as higher total numbers.

FIG. 16 shows increased reactivity with TD conditions in some cases (ie: OV7), suggesting that it might be beneficial to include TD in certain cases. FIGS. 18 and 19 show TILs grown with JAB or JAB TD exhibit a higher number of T cells specific for cancer-associated antigens Especially donor Ce4 shows an increase of these T cell populations when TME stimulators were added, compared to the IL-2 condition. It has been shown that a higher number and frequency of (neo)antigen-specific T cells in the infusion product is correlated with a better survival after TIL infusion (Heeke, C. et al., Neoantigen-reactive CD8+ T cells affect clinical outcome of adoptive cell therapy with tumor-infiltrating lymphocytes in melanoma. J Clin Invest, 132(2)).

FIGS. 20-22 show that adding JAB with a time delay of 96 h instead of 48 h or no time delay leads to similar total numbers of cells and also to similar higher frequencies and numbers of CD3+ and CD8+ T cells compared to the other TME stimulator combinations, which shows that the time delay of 96 h can be applied without compromising cell expansion.

FIG. 23 shows that the JAB TD of 96 h leads to an increased percentage of CD8+ T cells expressing activation/exhaustion markers BTLA, LAG3 and TIM3 compared to the other TME stimulator conditions while CD28 expression stays similar, which indicates the expansion of more activated but maybe exhausted cells.

FIG. 24 shows that by adding TME stimulators with a 96 h time delay, the population of Temra cells increases again to levels in the IL-2 samples, which can point towards more activated but exhausted cells in these samples.

FIG. 25 shows that the frequencies and numbers of the CD39-CD69− population is similar in all TME stimulator samples.

A further aspect of the present invention relates to expanded tumor infiltrating lymphocytes (TILs) for use in promoting regression of a cancer in a subject with cancer, the regression comprising the steps of: a) culturing autologous T cells by obtaining a first population of TILs from a tumor resected from a mammal, b) performing a depletion of suppressive cells, including regulatory T cells, and/or blocking negative signals by the addition of one or more TME stimulators from the group of “Inhibitors” to obtain a depleted population of TILs with or without the addition of “cytokines”, c) performing a first expansion by culturing the depleted population of TILs in a cell culture medium comprising: one or more TME stimulators from the group of “cytokines”, and/or one or more of the TME stimulators from the “Stimulator” group to produce a second population of TILs, d) performing a second expansion by supplementing the cell culture medium of the second population of TILs with additional IL-2 and/or other cytokines from the “cytokine” group, anti-CD3 antibody, and antigen presenting cells (APCs), to produce a third population of TILs, wherein the third population of TILs is a therapeutic population; and e) after administering nonmyeloablative lymphodepleting chemotherapy, administering to the mammal the therapeutic population of T cells, wherein the T cells administered to the mammal with or without IL-2 treatment, whereupon the regression of the cancer in the mammal is promoted.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition e.g., in the case of a vaccine.

The term “anti-CD3 antibody” refers to an antibody or variant thereof e.g., a monoclonal antibody and including human, humanized, chimeric or murine antibodies which are directed against the CD3 receptor in the T-cell antigen receptor of mature T cells. Anti-CD3 antibodies include OKT3, also known as muromonab. Anti-CD3 antibodies also include the UHCT1 clone, also known as T3 and CD3e. Other anti-CD3 antibodies include, for example, otelixizumab, teplizumab, and visilizumab. In an embodiment, the cell culture medium comprises OKT3 antibody. In some embodiments, the cell culture medium comprises about 30 ng/mL of OKT3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 12 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT3 antibody. In some embodiments, the cell culture medium does not comprise OKT3 antibody. Cytokines can be added in 0.1 ng/mL-10 ng/mL, 1 ng/mL-100 ng/mL, or in 1-100 ng/mL.

The term “IL-2” (also referred to herein as “IL2”) refers to the T-cell growth factor known as interleukin-2, and includes all forms of IL-2 including human and mammalian forms, conservative amino acid substitutions, glycoforms, biosimilars, and variants thereof.

After preparation of the tumor fragments, the resulting cells (i.e., fragments) are cultured in media containing IL-2 under conditions that favor the growth of TILs over tumor and other cells. In some embodiments, the tumor digests are incubated in e.g. 2 mL wells in media comprising inactivated human AB serum (or, in some cases, as outlined herein, in the presence of aAPC cell population) with 6000 IU/mL of IL-2. This primary cell population is cultured for a period of days, generally from 6 to 14 days, resulting in a bulk TIL population, generally about 1×10⁶ to 1×10⁸ bulk TIL cells. In some embodiments, the growth media during the first expansion comprises IL-2 or a variant thereof. In some embodiments, the IL-2 is recombinant human IL-2 (rhlL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the IL-2 stock solution is prepare as described in the examples. In some embodiments, the first expansion culture media comprises about 10,000 IU/mL of IL-2, about 9,000 IU/mL of IL-2, about 8,000 IU/mL of IL-2, about 7,000 IU/mL of IL-2, about 6000 IU/mL of IL-2 or about 5,000 IU/mL of IL-2. In some embodiments, the first expansion culture media comprises about 9,000 IU/mL of IL-2 to about 5,000 IU/mL of IL-2. In some embodiments, the first expansion culture media comprises about 8,000 IU/mL of IL-2 to about 6,000 IU/mL of IL-2. In some embodiments, the first expansion culture media comprises about 7,000 IU/mL of IL-2 to about 6,000 IU/mL of IL-2. In some embodiments, the first expansion culture media comprises about 6,000 IU/mL of IL-2. In an embodiment, the cell culture medium further comprises IL-2. In some embodiments, the cell culture medium comprises about 3000 IU/mL of IL-2. In an embodiment, the cell culture medium further comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or about 8000 IU/mL of IL-2.

IL-2, IL-4, IL-7, IL-12, IL-15, and/or IL-21 can also be added to step (b) and/or (c) of the present methods. Sometimes this is also done in step (e). A preferred embodiment relates to IL-2 to be added to step (b) and/or (c) of the present methods. These are part of the definition “cytokines” and part of the group of “cytokines” mentioned herein. The term “IL-4” (also referred to herein as “IL4”) refers to the cytokine known as interleukin 4, which is produced by Th2 T cells and by eosinophils, basophils, and mast cells. IL-4 regulates the differentiation of naive helper T cells (ThO cells) to Th2 T cells. The term “IL-7” (also referred to herein as “IL7”) refers to a glycosylated tissue-derived cytokine known as interleukin 7, which may be obtained from stromal and epithelial cells, as well as from dendritic cells. The term “IL-15” (also referred to herein as “IL15”) refers to the T cell growth factor known as interleukin-15, and includes all forms of IL-15 including human and mammalian forms, conservative amino acid substitutions, glycoforms, biosimilars, and variants thereof. The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. Interleukin 12 (IL-12) is an interleukin that is naturally produced by dendritic cells, macrophages, neutrophils, and human B-lymphoblastoid cells (NC-37) in response to antigenic stimulation. The term “IL-12” (also referred to herein as “IL12”) refers to the pleiotropic cytokine protein known as interleukin-12, and includes all forms of IL-12 including human and mammalian forms, conservative amino acid substitutions, glycoforms, biosimilars, and variants thereof.

Another aspect of the present invention relates to a method for expanding tumor infiltrating lymphocytes (TILs) into a therapeutic population of TILs comprising: a) culturing autologous T cells by obtaining a first population of TILs from a tumor resected from a mammal, b) performing a depletion of suppressive cells, including regulatory T cells, and/or blocking negative signals by the addition of one or more TME stimulators from the group of “Inhibitors” to obtain a depleted population of TILs with or without the addition of “cytokines”, c) performing a first expansion by culturing the depleted population of TILs in a cell culture medium comprising: one or more TME stimulators from the group of “cytokines”, and/or one or more of the TME stimulators from the “Stimulator” group to produce a second population of TILs, d) performing a second expansion by supplementing the cell culture medium of the second population of TILs with additional IL-2 and/or other cytokines from the “cytokine” group, anti-CD3 antibody, and antigen presenting cells (APCs), to produce a third population of TILs, wherein the third population of TILs is a therapeutic population.

The one or more TME stimulators from the group of “cytokines” can be added in step b). IL-2, IL-4, IL-7, IL-12, IL-15, and/or IL-21, as defined above are “cytokines”.

An embodiment of the present invention relates to the uses and methods of the present invention, wherein the group of “Inhibitors” that function by antagonizing/inhibiting receptors expressed on T cells (or their ligands) known to cause T-cell downregulation/deactivation/exhaustion. The group of “Inhibitors” can be selected from the group consisting one or more of: A) substances that act through the PD-1 receptor on T cells or its ligands PD-L1 or PD-L2, B) substances that act through the CTLA-4 receptor on T cells, C) substances that act through the LAG-3 receptor on T cells, D) substances that act through the TIGIT/CD226 receptor on T cells, E) substances that act through the KIR receptor on T-cells, F) substances that act through the TIM-3 receptor on T cells, G) substances that act through the BTLA receptor on T cells, and H) substances that act through the A2aR receptor on T-cells.

An embodiment of the present invention relates to the uses and methods of the present invention, wherein the group of “Inhibitors” are selected from the group consisting one or more of: A) substances that act through the PD-1 receptor on T cells, B) substances that act through the CTLA-4 receptor on T cells, C) substances that act through the LAG-3 receptor on T cells, and D) substances that act through the TIGIT/CD226 receptor on T cells.

An embodiment of the present invention relates to the uses and methods of the present invention, wherein group of “Inhibitors” are: A: substances that act through the PD-1 receptor on T cells, and B: substances that act through the CTLA-4 receptor on T cells.

An embodiment of the present invention relates to the uses and methods of the present invention, wherein the group of “Inhibitors” are: A: substances that act through the PD-1 receptor on T cells, B: substances that act through the CTLA-4 receptor on T cells, and C) substances that act through the LAG-3 receptor on T cells.

An embodiment of the present invention relates to the uses and methods of the present invention, wherein the group of “Inhibitors” are: A: substances that act through the PD-1 receptor on T cells, B: substances that act through the CTLA-4 receptor on T cells, and D) substances that act through the TIGIT/CD226 receptor on T cells.

An embodiment of the present invention relates to the uses and methods of the present invention, wherein the group of “Inhibitors” are: A) substances that act through the PD-1 receptor on T cells, B) substances that act through the CTLA-4 receptor on T cells, C) substances that act through the LAG-3 receptor on T cells, and D) substances that act through the TIGIT/CD226 receptor on T cells.

An embodiment of the present invention relates to the uses and methods of the present invention, wherein the group of “Inhibitors” are selected from the group consisting one or more of: P) substance that act through the molecule IDO, Q) substances that act through the TGFβ molecule TGFβ receptor on T cells, R) substances that act through the IL-10 molecule or IL-10 receptor on T cells, and S) substances that act through the IL-35 molecule or IL-35 receptor on T-cells. This group work by “Soluble inhibition” by antagonizing/inhibiting soluble molecules and cytokines and their receptors known to cause T-cell downregulation/deactivation/exhaustion.

An embodiment of the present invention relates to the uses and methods of the present invention, wherein the group of “Inhibitors” are selected from the group consisting of one or more of: T) cyclophosphamides, U) TKIs, V) substances that act through αCD25, and X) IL2/Diphteria toxin fusions. This group works by adding factors known to downregulate and/or deplete regulatory T cells thereby favoring ex-vivo effector T-cell expansion.

An embodiment of the present invention relates to the uses and methods of the present invention, wherein the group of “Stimulator” are selected from the group consisting one or more of: 1) substances that act through the OX40/CD134 receptor on T cells, J) substances that act through the 4-1 BB/CD137 receptor on T cells, K) substances that act through the CD28 receptor on T cells, L) substances that act through the ICOS receptor on T cells, M) substances that act through the GITR receptor on T cells, N) substances that act through the CD40L receptor on T cells, O) substances that act through the CD27 receptor on T cells, and W) substances that act through CD-3. These are “Stimulators” that work by agonizing/stimulating receptors expressed on T cells known to cause T-cell upregulation/activation/reinvigoration

An embodiment of the present invention relates to the uses and methods of the present invention, wherein the group of “Stimulator” is: J) substances that act through the 4-1 BB/CD137 receptor on T cells.

An embodiment of the present invention relates to the uses and methods of the present invention, wherein: the group of “Inhibitors” in step b) are: A: substances that act through the PD-1 receptor on T cells, and B: substances that act through the CTLA-4 receptor on T cells, and wherein the group of “Stimulator” in step c) is: J) substances that act through the 4-1BB/CD137 receptor on T cells. One or more cytokines can be added to steps b) and/or c). These embodiments are shown experimentally in examples 1-2.

An aspect of the present invention relates to including a time lapse between steps b) and c), i.e. where the two steps are performed with a certain time period apart.

Thus, embodiment of the present invention relates to the uses and methods of the present invention, wherein the step b) and step c) are performed 1-2 days apart. An embodiment of the present invention relates to the uses and methods of the present invention, wherein the step b) and step c) are performed 1-3 days apart. An embodiment of the present invention relates to the uses and methods of the present invention, wherein the step b) and step c) are performed 1-4 days apart. An embodiment of the present invention relates to the uses and methods of the present invention, wherein the step b) and step c) are performed 1-5 days apart. An embodiment of the present invention relates to the uses and methods of the present invention, wherein the step b) and step c) are performed 1-6 days apart. An embodiment of the present invention relates to the uses and methods of the present invention, wherein the step b) and step c) are performed 1-7 days apart.

An embodiment of the present invention relates to the uses and methods of the present invention, wherein the step b) and step c) are performed 1-8 days apart. An embodiment of the present invention relates to the uses and methods of the present invention, wherein the step b) and step c) are performed 2-8 days apart. An embodiment of the present invention relates to the uses and methods of the present invention, wherein the step b) and step c) are performed 3-8 days apart. An embodiment of the present invention relates to the uses and methods of the present invention, wherein the step b) and step c) are performed 4-8 days apart. An embodiment of the present invention relates to the uses and methods of the present invention, wherein the step b) and step c) are performed 5-8 days apart. An embodiment of the present invention relates to the uses and methods of the present invention, wherein the step b) and step c) are performed 6-8 days apart. An embodiment of the present invention relates to the uses and methods of the present invention, wherein the step b) and step c) are performed 7-8 days apart. An embodiment of the present invention relates to the uses and methods of the present invention, wherein the step b) and step c) are performed 2-7 days apart. An embodiment of the present invention relates to the uses and methods of the present invention, wherein the step b) and step c) are performed 3-7 days apart. An embodiment of the present invention relates to the uses and methods of the present invention, wherein the step b) and step c) are performed 4-7 days apart. An embodiment of the present invention relates to the uses and methods of the present invention, wherein the step b) and step c) are performed 5-7 days apart. An embodiment of the present invention relates to the uses and methods of the present invention, wherein the step b) and step c) are performed 6-7 days apart. An embodiment of the present invention relates to the uses and methods of the present invention, wherein the step b) and step c) are performed 2-6 days apart. An embodiment of the present invention relates to the uses and methods of the present invention, wherein the step b) and step c) are performed 3-6 days apart. An embodiment of the present invention relates to the uses and methods of the present invention, wherein the step b) and step c) are performed 4-6 days apart. An embodiment of the present invention relates to the uses and methods of the present invention, wherein the step b) and step c) are performed 5-6 days apart. An embodiment of the present invention relates to the uses and methods of the present invention, wherein the step b) and step c) are performed 2-5 days apart. An embodiment of the present invention relates to the uses and methods of the present invention, wherein the step b) and step c) are performed 3-5 days apart. An embodiment of the present invention relates to the uses and methods of the present invention, wherein the step b) and step c) are performed 4-5 days apart. An embodiment of the present invention relates to the uses and methods of the present invention, wherein the step b) and step c) are performed 2-4 days apart. An embodiment of the present invention relates to the uses and methods of the present invention, wherein the step b) and step c) are performed 3-4 days apart. An embodiment of the present invention relates to the uses and methods of the present invention, wherein the step b) and step c) are performed on two consecutive days. This means that step b) for example can be performed during a given day (for example a workday), and then step c) is performed the next day (for example the next workday). This means that the time delay (TD) or time lapse can be less than 1 day, for example 20 hours, 18-24 hours, or 14-20 hours.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) of the patient prior to the introduction of the TILs of the invention.

The methods of the present invention, from step (a) to step (d), can be performed in a closed system. The term “closed system” refers to a system that is closed to the outside environment. Any closed system appropriate for cell culture methods can be employed with the methods of the present invention. Closed systems include, for example, but are not limited to closed G-Rex containers.

The term “TME stimulators” relates to substances (or agents) that have the ability to create a favorable microenvironment within the tumor where exhausted T cells can be reinvigorated in order to expand many fold and restore their anti-tumor functionality. Thus, in one or more embodiments, the one or more TME stimulators are selected from the groups consisting of: (x) one or more substances that are capable of antagonizing and/or inhibiting receptors expressed on T cells (or their ligands) known to cause T-cell downregulation, deactivation and/or exhaustion, (y) one or more substances that are capable of agonizing and/or stimulating receptors expressed on T cells known to cause T-cell upregulation, activation, and/or reinvigoration, (z) one or more substances that are capable of antagonizing and/or inhibiting soluble molecules and cytokines and their receptors known to cause T-cell downregulation, deactivation, and/or exhaustion, and (v) one or more substances that are capable of downregulating and/or depleting suppressive cells, including regulatory T cells, thereby favoring ex-vivo effector T-cell expansion, and (w) one or more substances from the groups (x), (y), (z) and/or (v). Group (w) can be one, two or three of the substances from (x), (y), (z) and/or (v). In one or more embodiments, (w) is one or two of the substances from (x). In one or more embodiments, (w) is one or two of the substances from (y). In one or more embodiments, (w) is one or two of the substances from (z). In one or more embodiments, (w) is one or two of the substances from (v).

In one or more embodiments, the substances that are capable of antagonizing and/or inhibiting receptors expressed on T cells (or their ligands) known to cause T-cell downregulation, deactivation and/or exhaustion are selected from the groups consisting of: A: substances that act through the PD-1 receptor on T cells, B: substances that act through the CTLA-4 receptor on T cells, C: substances that act through the LAG-3 receptor on T cells, D: substances that act through the TIGIT/CD226 receptor on T cells, E: substances that act through the KIR receptor on T cells, F: substances that act through the TIM-3 receptor on T cells, G: substances that act through the BTLA receptor on T cells, and H: substances that act through the A2aR receptor on T cells. It is to be understood that the definition of substances that act through a given receptor also can cover the same receptors ligand. This means e.g. that for the PD-1 receptor, substances that target the PD-L1 or PD-L2 can also be covered. Group A can therefore cover substances that act through the PD-1 receptor on T cells as well as its ligand(s).

The substances of the present invention can be an antibody. The substances of the present invention can be a peptide. The substances of the present invention can be a small molecule.

The term “antibody” refers to an antibody or variant thereof e.g., a monoclonal antibody including human, humanized, chimeric, or murine antibodies, or F(ab′)2 or Fab fragment, or Nanobody.

In one or more embodiments, the substance of group A is an antibody selected from one or more from the group consisting of pembrolizumab, nivolumab, cemiplimab, sym021, atezolizumab, avelumab, durvalumab, Toripalimab, Sintilimab, Camrelizumab, Tislelizumab, Sasanlimab, and Dostarlimab. In one or more embodiments, the substance of group A is a small molecule selected from one or more from the group consisting of MAX-10181, YPD-29B, IMMH-010, INCB086550, GS-4224, DPPA-1, TPP-1, BMS-202, CA-170, JQ1, eFT508, Osimertinib, PlatycodinD, PD-LYLSO, Curcumin, and Metformin. In one or more embodiments, the substance of group A is selected from one or more from the group consisting of pembrolizumab, nivolumab, cemiplimab, sym021, atezolizumab, avelumab, durvalumab, Toripalimab, Sintilimab, Camrelizumab, Tislelizumab, Sasanlimab, Dostarlimab, MAX-10181, YPD-29B, IMMH-010, INCB086550, GS-4224, DPPA-1, TPP-1, BMS-202, CA-170, JQ1, eFT508, Osimertinib, PlatycodinD, PD-LYLSO, Curcumin, and Metformin. In one or more embodiments, the substance of group A is pembrolizumab. In one or more embodiments, the substance of group A is nivolumab. In one or more embodiments, the substance of group A is cemiplimab. In one or more embodiments, the substance of group A is sym021. In one or more embodiments, the substance of group A is atezolizumab. In one or more embodiments, the substance of group A is avelumab. In one or more embodiments, the substance of group A is durvalumab. In one or more embodiments, the substance of group A is Toripalimab. In one or more embodiments, the substance of group A is Sintilimab. In one or more embodiments, the substance of group A is Camrelizumab. In one or more embodiments, the substance of group A is Tislelizumab. In one or more embodiments, the substance of group A is Sasanlimab. In one or more embodiments, the substance of group A is Dostarlimab. In one or more embodiments, the substance of group A is MAX-10181. In one or more embodiments, the substance of group A is YPD-29B. In one or more embodiments, the substance of group A is IMMH-010. In one or more embodiments, the substance of group A is INCB086550. In one or more embodiments, the substance of group A is GS-4224. In one or more embodiments, the substance of group A is DPPA-1. In one or more embodiments, the substance of group A is TPP-1. In one or more embodiments, the substance of group A is BMS-202. In one or more embodiments, the substance of group A is CA-170. In one or more embodiments, the substance of group A is JQ1. In one or more embodiments, the substance of group A is eFT508. In one or more embodiments, the substance of group A is Osimertinib. In one or more embodiments, the substance of group A is PlatycodinD. In one or more embodiments, the substance of group A is PD-LYLSO. In one or more embodiments, the substance of group A is Curcumin. In one or more embodiments, the substance of group A is Metformin.

In one embodiment group A is a substance that acts through the PD-1 receptor by blocking the interaction with its ligand including PD-L1/PD-L2. In one embodiment group A is a substance that acts through the PD-L1/L2 by blocking the interaction with its receptor including PD-1. In one embodiment group A is a substance that blocks the interaction between PD-1 and PD-L1/PD-L2. In one embodiment group A is a substance that blocks the signaling of the PD-1 receptor and/or its downstream signaling pathways. In one embodiment group A is a substance that downregulates the expression of PD-L1/PD-L2. In one embodiment group A is a substance that promotes degradation of PD-L1/PD-L2.

In one or more embodiments, the substance of group B is selected from one or more antibodies from the group consisting of ipilimumab and tremelimumab. In one or more embodiments, the substance of group B is ipilimumab. In one or more embodiments, the substance of group B is tremelimumab.

In one embodiment group B is a substance that acts through the CTLA4 receptor by blocking the interaction with its ligand including B7-1 or B7-2. In one embodiment group B is a substance that acts through the B7-1 or B7-2 by blocking the interaction with its receptor including CTLA4. In one embodiment group B is a substance that blocks the interaction between CTLA4 and B7-1 or B7-2. In one embodiment group B is a substance that blocks the signaling of the B7-1 or B7-2 receptor and/or its downstream signaling pathways. In one embodiment group B is a substance that blocks the signaling of the CTLA4 receptor and/or its downstream signaling pathways. In one embodiment group B is a substance that induces cell death upon binding the CTLA4 receptor. In one embodiment group B is an antibody with antibody-dependent cellular cytotoxicity (ADCC). In one embodiment group B is a substance that depletes CTLA4 expressing cells. In one embodiment group B is a substance that depletes regulatory T cells through binding CTLA4 and killing the cell. In one embodiment group B is a substance capable of blocking the interaction of CTLA4 and its ligand, and mediating cell specific cytotoxicity through binding to CTLA4.

In one or more embodiments, the substance of group C is selected from one or more from the group consisting of relatlimab, eftilagimo alpha, sym022, BMS-986016, and GSK28-31781.

In one embodiment group C is a substance that acts through the LAG3 receptor by blocking the interaction with its receptor. In one embodiment group C is a substance that blocks the signaling of the LAG3 receptor and/or its downstream signaling pathways.

In one or more embodiments, the substance of group D is tiragolumab or Liothyronine. In one or more embodiments, the substance of group D is tiragolumab. In one or more embodiments, the substance of group D is Liothyronine.

In one embodiment group D is a substance that acts through the TIGIT receptor by blocking the interaction with its receptor. In one embodiment group D is a substance that blocks the signaling of the TIGIT receptor and/or its downstream signaling pathways. In one embodiment group D is a substance that induces cell death upon binding the TIGIT receptor. In one embodiment group D is an antibody with antibody-dependent cellular cytotoxicity (ADCC). In one embodiment group D is a substance capable of blocking the interaction of TIGIT and its ligand, and mediating cell specific cytotoxicity through binding to TIGIT.

In one or more embodiments, the substance of group E is lirilumab. In one or more embodiments, the substance of group F is sym023. In one or more embodiments, the substance of group G is 40E4 and PJ196.

In one or more embodiments, the substances that are capable of agonizing and/or stimulating receptors expressed on T cells known to cause T-cell upregulation, activation, and/or reinvigoration are selected from the groups consisting of: I: substances that act through the OX40/CD134 receptor on T cells, J: substances that act through the 4-1 BB/CD137 receptor on T cells, K: substances that act through the CD28 receptor on T cells, L: substances that act through the ICOS receptor on T cells, M: substances that act through the GITR receptor on T cells, N: substances that act through the CD40L receptor on T cells, and O: substances that act through the CD27 receptor on T cells.

In one or more embodiments, the substance of group J is selected from one or more antibodies from the group consisting of urelumab and utomilumab. In one or more embodiments, the substance of group J is selected from one or more peptides from the group consisting of BCY7835, and BCY7838 from Bicycle Therapeutics. In one or more embodiments, the substance of group J is selected from one or more from the group consisting of BCY7835, BCY7838, urelumab and utomilumab. In one or more embodiments, the substance of group J is urelumab. In one or more embodiments, the substance of group J is utomilumab. In one or more embodiments, the substance of group J is BCY7835. In one or more embodiments, the substance of group J is BCY7838.

In one embodiment group J is a substance that act through the 4-1 BB/CD137 receptor. In one embodiment group J is a substance that act through the 4-1 BB/CD137 receptor and stimulates the growth of T cells. In one embodiment group J is a substance that act through the 4-1 BB/CD137 receptor and stimulates antigen presenting cells (APC).

The group J substances can be used in combination with an anti-CD3 substance such as OKT-3. One combination can therefore be urelumab and OKT-3 (urelumab/OKT-3). Another combination can be utomilumab and OKT-3 (utomilumab/OKT-3). An anti-CD3 substances, such as OKT-3, belongs to group W as defined herein. In one embodiment group W is a substance that binds and activates CD3 on T cells. In one or more embodiments, the substance of group K is theralizumab. In one or more embodiments, the substance of group 0 is valilumab.

In one or more embodiments, one or more of the substances of group A can be combined with one or more of the substances of group B. In one or more embodiments, one or more of the substances of group A can be combined with one or more of the substances of group B, and with one or more of the substances of group J. These combinations are shown to be effective in the examples of the present disclosure. This means that one or more substances of group A selected from one or more from the group consisting of pembrolizumab, nivolumab, cemiplimab, sym021, atezolizumab, avelumab can be combined with one or more of the substances of group B which is selected from one or more from the group consisting of ipilimumab and tremelimumab. These can then be combined with one or more substances of group J which is selected from one or more from the group consisting of urelumab and utomilumab. The group J substances can be used in combination with an anti-CD3 substance such as OKT-3. One combination can therefore be one or more substances of group A selected from one or more from the group consisting of pembrolizumab, nivolumab, cemiplimab, sym021, atezolizumab, avelumab combined with ipilimumab from group B and urelumab from group J. A specific selection can be pembrolizumab combined with ipilimumab from group B and urelumab from group J, with or without an anti-CD3 substance such as OKT-3.

In one or more embodiments, the substances that are capable of antagonizing and/or inhibiting soluble molecules and cytokines and their receptors known to cause T-cell downregulation, deactivation, and/or exhaustion are selected from the groups consisting of: P: substances that act through the IDO1/2 receptor on T cells, Q: substances that act through the TGFβ receptor on T cells, R: substances that act through the IL-10 receptor on T cells, and S: substances that act through the IL-35 receptor on T cells.

In one or more embodiments, the substance of group P is epacedostat. In one or more embodiments, the substance of group Q is linrodostat. In one or more embodiments, the substance of group R is galunisertib.

In one or more embodiments, the substances that are capable of downregulating and/or depleting suppressive cells, including regulatory T cells, thereby favoring ex-vivo effector T-cell expansion are selected from the groups consisting of: T: cyclophosphamides, U: TKIs, V: substances that act through αCD25, and X: IL2/Diphteria toxin fusions.

In one or more embodiments, the substance of group U is sunitinib. In one or more embodiments, the substance of group V is selected from one or more from the group consisting of sorafenib, imatinib and daclizumab. In one or more embodiments, the substance of group X is dinileukin diftitox.

Using the approaches presented herein allows for dose levels that are much higher than would be tolerated in vivo. The concentrations can therefore be at least twice as high as the maximum allowed dose tolerated in vivo. The concentration can be even higher such as 5-10 times as high as the maximum allowed dose tolerated in vivo. Thus, in one or more embodiments, the concentration of substance in is 0.1 μg/mL to 300 μg/mL. The concentration can also be 1 μg/mL to 100 μg/mL. The concentration can also be 10 μg/mL to 100 μg/mL. The concentration can also be 1 μg/mL to 10 μg/mL.

In one or more embodiments, the therapeutic population of T cells is used to treat a cancer type selected from the groups consisting of: 1: solid tumors, 2: ICI naive tumors, 3: MSI-H tumors, 4: Hematological tumors, 5: Hyper-mutated tumors (such as POL-E and POL-D mutated tumors), and 6: virus-induced tumors.

In one or more embodiments, the therapeutic population of T cells is used to treat a cancer type selected from the groups consisting of breast cancer, renal cell cancer, bladder cancer, melanoma, cervical cancer, gastric cancer, colorectal cancer, lung cancer, head and neck cancer, ovarian cancer, Hodgkin lymphoma, pancreatic cancer, liver cancer, and sarcomas.

In one or more embodiments, the therapeutic population of T cells is used to treat a breast cancer. In one or more embodiments, the therapeutic population of T cells is used to treat renal cell cancer. In one or more embodiments, the therapeutic population of T cells is used to treat bladder cancer. In one or more embodiments, the therapeutic population of T cells is used to treat melanoma. In one or more embodiments, the therapeutic population of T cells is used to treat cervical cancer. In one or more embodiments, the therapeutic population of T cells is used to treat gastric cancer. In one or more embodiments, the therapeutic population of T cells is used to treat colorectal cancer. In one or more embodiments, the therapeutic population of T cells is used to treat lung cancer. In one or more embodiments, the therapeutic population of T cells is used to treat head and neck cancer. In one or more embodiments, the therapeutic population of T cells is used to treat ovarian cancer. In one or more embodiments, the therapeutic population of T cells is used to treat Hodgkin lymphoma. In one or more embodiments, the therapeutic population of T cells is used to treat pancreatic cancer. In one or more embodiments, the therapeutic population of T cells is used to treat liver cancer. In one or more embodiments, the therapeutic population of T cells is used to treat sarcomas.

In one or more embodiments, steps (a) through (c), (d) or (e) are performed within a period of about 20 days to about 45 days. In one or more embodiments, steps (a) through (c) or (d) are performed within a period of about 20 days to about 40 days. In one or more embodiments, steps (a) through (c) or (d) are performed within a period of about 25 days to about 40 days. In one or more embodiments, steps (a) through (c) or (d) are performed within a period of about 30 days to about 40 days. In one or more embodiments, steps (a) through (b) are performed within a period of about 10 days to about 28 days. In one or more embodiments, steps (a) through (b) are performed within a period of about 10 days to about 20 days.

In some embodiments, the depletion step (step (b)), can be performed for a period of 1-7 days. This period can also be 1-3 days, 1-2 days, or 4-7 days.

In some embodiments, the first TIL expansion can proceed for 1 day, 2 days, 3 days, 4 days, 5 days, 6 days, 7 days, 8 days, 9 days, 10 days, 11 days, 12 days, 13 days, or 14 days. In some embodiments, the first TIL expansion can proceed for 1 day to 14 days. In some embodiments, the first TIL expansion can proceed for 2 days to 14 days. In some embodiments, the first TIL expansion can proceed for 3 days to 14 days. In some embodiments, the first TIL expansion can proceed for 4 days to 14 days. In some embodiments, the first TIL expansion can proceed for 5 days to 14 days. In some embodiments, the first TIL expansion can proceed for 6 days to 14 days. In some embodiments, the first TIL expansion can proceed for 7 days to 14 days. In some embodiments, the first TIL expansion can proceed for 8 days to 14 days. In some embodiments, the first TIL expansion can proceed for 9 days to 14 days. In some embodiments, the first TIL expansion can proceed for 10 days to 14 days. In some embodiments, the first TIL expansion can proceed for 11 days to 14 days. In some embodiments, the first TIL expansion can proceed for 12 days to 14 days. In some embodiments, the first TIL expansion can proceed for 13 days to 14 days. In some embodiments, the first TIL expansion can proceed for 14 days. In some embodiments, the first TIL expansion can proceed for 1 day to 11 days. In some embodiments, the first TIL expansion can proceed for 2 days to 11 days. In some embodiments, the first TIL expansion can proceed for 3 days to 11 days. In some embodiments, the first TIL expansion can proceed for 4 days to 11 days. In some embodiments, the first TIL expansion can proceed for 5 days to 11 days. In some embodiments, the first TIL expansion can proceed for 6 days to 11 days. In some embodiments, the first TIL expansion can proceed for 7 days to 11 days. In some embodiments, the first TIL expansion can proceed for 8 days to 11 days. In some embodiments, the first TIL expansion can proceed for 9 days to 11 days. In some embodiments, the first TIL expansion can proceed for 10 days to 11 days. In some embodiments, the first TIL expansion can proceed for 11 days.

In one or more embodiments, step (c) is performed within a period of about 6 days to about 18 days. In one or more embodiments, step (c) is performed within a period of about 7 days to about 14 days. In one or more embodiments, step (c) is performed within a period of about 7 days to about 10 days. In one or more embodiments, step (c) is performed within a period of about 6 days to about 12 days.

In one or more embodiments, step (d) is performed within a period of about 12 days to about 18 days. In one or more embodiments, step (d) is performed within a period of about 10 days to about 28 days. In one or more embodiments, step (d) is performed within a period of about 10 days to about 20 days. In one or more embodiments, step (d) is performed within a period of about 12 days to about 18 days.

In some embodiments, the transition from the first expansion to the second expansion occurs at 1 day, 2 days, 3 days, 4 days, 5 days, 6 days, 7 days, 8 days, 9 days, 10 days, 11 days, 12 days, 13 days, or 14 days from when fragmentation occurs. In some embodiments, the transition from the first expansion to the second expansion occurs 1 day to 14 days from when fragmentation occurs. In some embodiments, the first TIL expansion can proceed for 2 days to 14 days. In some embodiments, the transition from the first expansion to the second expansion occurs 3 days to 14 days from when fragmentation occurs. In some embodiments, the transition from the first expansion to the second expansion occurs 4 days to 14 days from when fragmentation occurs. In some embodiments, the transition from the first expansion to the second expansion occurs 5 days to 14 days from when fragmentation occurs. In some embodiments, the transition from the first expansion to the second expansion occurs 6 days to 14 days from when fragmentation occurs. In some embodiments, the transition from the first expansion to the second expansion occurs 7 days to 14 days from when fragmentation occurs. In some embodiments, the transition from the first expansion to the second expansion occurs 8 days to 14 days from when fragmentation occurs. In some embodiments, the transition from the first expansion to the second expansion occurs 9 days to 14 days from when fragmentation occurs. In some embodiments, the transition from the first expansion to the second expansion occurs 10 days to 14 days from when fragmentation occurs. In some embodiments, the transition from the first expansion to the second expansion occurs 11 days to 14 days from when fragmentation occurs. In some embodiments, the transition from the first expansion to the second expansion occurs 12 days to 14 days from when fragmentation occurs. In some embodiments, the transition from the first expansion to the second expansion occurs 13 days to 14 days from when fragmentation occurs. In some embodiments, the transition from the first expansion to the second expansion occurs 14 days from when fragmentation occurs. In some embodiments, the transition from the first expansion to the second expansion occurs 1 day to 11 days from when fragmentation occurs. In some embodiments, the transition from the first expansion to the second expansion occurs 2 days to 11 days from when fragmentation occurs. In some embodiments, the transition from the first expansion to the second expansion occurs 3 days to 11 days from when fragmentation occurs. In some embodiments, the transition from the first expansion to the second expansion occurs 4 days to 11 days from when fragmentation occurs. In some embodiments, the transition from the first expansion to the second expansion occurs 5 days to 11 days from when fragmentation occurs. In some embodiments, the transition from the first expansion to the second expansion occurs 6 days to 11 days from when fragmentation occurs. In some embodiments, the transition from the first expansion to the second expansion occurs 7 days to 11 days from when fragmentation occurs. In some embodiments, the transition from the first expansion to the second expansion occurs 8 days to 11 days from when fragmentation occurs. In some embodiments, the transition from the first expansion to the second expansion occurs 9 days to 11 days from when fragmentation occurs. In some embodiments, the transition from the first expansion to the second expansion occurs 10 days to 11 days from when fragmentation occurs. In some embodiments, the transition from the first expansion to the second expansion occurs 11 days from when fragmentation occurs.

One of the key findings has been that more TILs can be generated faster. This has high value because there is a certain amount of cells that are needed in order to be relevant for medical treatment. More cells faster will drive down the costs for production and also provide treatment to the patient faster. In one or more embodiments, step (c) results in 1×10⁶ to 1×10⁷ cells, such as 2×10⁶ to 5×10⁶ cells. In one or more embodiments, step (c) results in 5×10⁶ to 1×10⁷ cells. In one or more embodiments, step (c) results in 1×10⁶ to 5×10⁷ cells. In one or more embodiments, step (c) results in 1×10⁷ to 5×10⁷ cells. In one or more embodiments, step (c) results in 1×10⁷ to 1×10¹² cells, such as 1×10⁸ to 5×10⁹ cells, such as 1×10⁹ to 5×10⁹ cells, such as 1×10⁸ to 5×10¹⁰ cells, such as 1×10⁹ to 5×10¹¹ cells. In one or more embodiments, step (c) results in an at least 10⁴ fold increase as compared to the number of cells after the expansion in step (c), such as at least 10³ fold increase, such as at least 10² fold increase, such as at least 10 fold increase. In one or more embodiments, step (d) results in 1×10⁷ to 1×10¹⁰ cells. In one or more embodiments, step (d) results in 1×10⁷ to 1×10⁹ cells. In one or more embodiments, step (d) results in 1×10⁷ to 1×10⁸ cells. In one or more embodiments, step (d) results in 1×10¹⁰ to 1×10¹¹ cells. In one or more embodiments, step (d) results in 1×10¹¹ to 2×10¹¹ cells. In one or more embodiments, step (d) results in at least 1×10¹¹ cells. In one or more embodiments, step (d) results in at least 2×10¹¹ cells.

In some embodiments, the antigen-presenting feeder cells (APCs) are PBMCs. In some embodiments, the antigen-presenting feeder cells (APCs) are allogeneic feeder cells. In some embodiments, the antigen-presenting feeder cells are artificial antigen-presenting feeder cells. In an embodiment, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to antigen-presenting feeder cells in the second expansion is between 1 to 50 and 1 to 300. In an embodiment, the ratio of TILs to antigen-presenting feeder cells in the second expansion is between 1 to 100 and 1 to 200. In one or more embodiments, the APCs are artificial APCs (aAPCs).

In an embodiment all of the TILs obtained in step c are transferred to step d and co-cultured with a fixed number of APCs. For example if 10-100 or 1-200 million cells are obtained in step c, all of them are transferred to step d and cultured with 4 billion APC. The ratio or cells to APCs can be 1:200. It could also be in increments, 10-50 million TILs are co-cultured with 4 billion APC's. 50-100 are co-cultured with 10 billion APC's.

In an embodiment the TILs obtained in step c, are cultures with APCs in a concentration of about 0,1-1 million cells/cm², of about 1 to 2 million cells/cm², of about 1 to 3 million cells/cm², of about 1 to 5 million cells/cm², of about 1 to 10 million cells/cm², of about 1 to 20 million cells/cm², of about 1 to 50 million cells/cm². The APCs can also be in a concentration of 1, 2, 3, 4 or 5 million cells/cm².

In an embodiment, TILs expanded using APCs of the present disclosure are administered to a patient as a pharmaceutical composition. In an embodiment, the pharmaceutical composition is a suspension of TILs in a sterile buffer. TILs expanded using PBMCs of the present disclosure may be administered by any suitable route as known in the art. In some embodiments, the T cells are administered as a single intra-arterial or intravenous infusion, which preferably lasts approximately 30 to 60 minutes. Other suitable routes of administration include intraperitoneal, intrathecal, intra-tumoral, and intralymphatic. In one or more embodiments, the therapeutic population of TILs are infused into a patient.

In one or more embodiments, the cells are removed from the cell culture and cryopreserved in a storage medium prior to performing step (d).

In one or more embodiments, the method further comprises the step of transducing the first population of TILs with an expression vector comprising a nucleic acid encoding a chimeric antigen receptor (CAR) comprising a single chain variable fragment antibody fused with at least one endodomain of a T-cell signaling molecule.

In one or more embodiments, step (c) further comprises a step of removing the cells from the cell culture medium.

In one or more embodiments, step (a) further comprises processing of the resected tumor into multiple tumor fragments, such as 4 to 50 fragments, such as 20 to 30 fragments. In one or more embodiments, the fragments have a size of about 1 to 50 mm³. In one or more embodiments, the fragments have a size of about 5 to 50 mm³. In one or more embodiments, the fragments have a size of about 0.1 to 10 mm³. In one or more embodiments, the fragments have a size of about 0.1 to 1 mm³. In one or more embodiments, the fragments have a size of about 0.5 to 5 mm³. In one or more embodiments, the fragments have a size of about 1 to 10 mm³. In one or more embodiments, the fragments have a size of about 1 to 3 mm³. The terms “fragmenting”, “fragment,” and “fragmented”, as used herein to describe processes for disrupting a tumor, includes mechanical fragmentation methods such as crushing, slicing, dividing, and morcellating tumor tissue as well as any other method for disrupting the physical structure of tumor tissue.

In one or more embodiments, the mammal is a human. In some embodiments, the TILs are obtained from tumor fragments. In some embodiments, the tumor fragment is obtained by sharp dissection. In some embodiments, the tumor fragment is between about 0.1 mm³ and 10 mm³. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumors are 1-4 mm×1-4 mm×1-4 mm. In some embodiments, the tumors are 1 mm×1 mm×1 mm. In some embodiments, the tumors are 2 mm×2 mm×2 mm. In some embodiments, the tumors are 3 mm×3 mm×3 mm. In some embodiments, the tumors are 4 mm×4 mm×4 mm. Currently fairly large fragment sizes are needed (more than 5 mm³). The present invention allows for the use of smaller fragments because the cells grow in a more optimized way reaching the cell count needed for treatment faster. The use of smaller fragments means that patients that until now have not been treatable because e.g. because their tumor has been too small or because it only has been possible to obtain a small tumor sample, now can be treated. The size of the fragments used in the methods of the present invention can therefore be important.

In some embodiments, the tumor fragmentation is performed in order to maintain the tumor internal structure. In some embodiments, the tumor fragmentation is performed without preforming a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In one or more embodiments, the cell culture medium is provided in a container selected from the group consisting of a G-Rex container and a Xuri cellbag.

An aspect relates to a population of tumor infiltrating lymphocytes (TILs) obtainable by a method of any of the previous claims.

A further aspect relates to expanded tumor infiltrating lymphocytes (TILs) for use in treating a subject with cancer, the treatment comprising the steps of: culturing autologous T cells by obtaining a first population of TILs from a tumor resected from a mammal performing a first expansion by culturing the first population of TILs in a cell culture medium comprising IL-2 and one or more TME stimulators to produce a second population of TILs; performing a second expansion by supplementing the cell culture medium of the second population of TILs with additional IL-2 and/or other cytokines from the “cytokine” group, anti-CD3, and antigen presenting cells (APCs), to produce a third population of TILs, wherein the third population of TILs is a therapeutic population; and after administering nonmyeloablative lymphodepleting chemotherapy, administering to the mammal the therapeutic population of T cells, wherein the T cells administered to the mammal, whereupon the regression of the cancer in the mammal is promoted.

In an embodiment, the invention includes a method of treating a cancer with a population of TILs, or use of the TILs to treat cancer, wherein a patient is pre-treated with non-myeloablative chemotherapy prior to an infusion of TILs according to the present disclosure. In an embodiment, the non-myeloablative chemotherapy is cyclophosphamide 60 mg/kg/d for 2 days (days 7 and 2 prior to TIL infusion) and fludarabine 25 mg/m2/d for 5 days (days 5 to 1 prior to TIL infusion). In an embodiment, after non-myeloablative chemotherapy and TIL infusion (at day 0) according to the present disclosure, the patient receives an intravenous infusion of IL-2 intravenously at 720,000 IU/kg every 8 hours to physiologic tolerance.

In an embodiment, the non-myeloablative chemotherapy is cyclophosphamide 500 mg/m2/day i.v. for 3 days on day −4, −3, −2 and fludarabine 30 mg/m2/day i.v. for 2 days on day −4, −3 followed by TIL infusion on day 0. In an embodiment, after non-myeloablative chemotherapy and TIL infusion (at day 0) according to the present disclosure, the patient receives an intravenous infusion of IL-2 intravenously at 720,000 IU/kg every 8 hours to physiologic tolerance.

In some embodiments, the present disclosure provides a method of treating a cancer with a population of tumor infiltrating lymphocytes (TILs) comprising the steps of (a) obtaining a first population of TILs from a tumor resected from a patient; b) performing a depletion of suppressive cells, including regulatory T cells, and/or blocking negative signals by the addition of one or more TME stimulators from the group of “Inhibitors” to obtain a depleted population of TILs with or without the addition of “cytokines”, (cc) performing an initial expansion of the first population of TILs in a first cell culture medium to obtain a second population of TILs, wherein the second population of TILs is at least 5-fold greater in number than the first population of TILs, and wherein the first cell culture medium comprises IL-2 and one or more TME stimulators; (d) performing a rapid expansion of the second population of TILs using a population of myeloid artificial antigen presenting cells (myeloid aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and anti-CD3; (e) administering a therapeutically effective portion of the third population of TILs to a patient with the cancer. In some embodiments, the present disclosure a population of tumor infiltrating lymphocytes (TILs) for use in treating cancer, wherein the population of TILs are obtainable by a method comprising the steps of b) performing a depletion of suppressive cells, including regulatory T cells, and/or blocking negative signals by the addition of one or more TME stimulators from the group of “Inhibitors” to obtain a depleted population of TILs with or without the addition of “cytokines”, (c) performing an initial expansion of a first population of TILs obtained from a tumor resected from a patient in a first cell culture medium to obtain a second population of TILs, wherein the second population of TILs is at least 5-fold greater in number than the first population of TILs, and wherein the first cell culture medium comprises IL-2; (d) performing a rapid expansion of the second population of TILs using a population of myeloid artificial antigen presenting cells (myeloid aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and anti-CD3; (e) administering a therapeutically effective portion of the third population of TILs to a patient with the cancer. In some embodiments, the method comprises a first step (a) of obtaining the first population of TILs from a tumor resected from a patient. In some embodiments, the IL-2 is present at an initial concentration of about 3000 IU/mL and anti-CD3antibody is present at an initial concentration of about 30 ng/mL in the second cell culture medium. In some embodiments, first expansion is performed over a period not greater than 14 days. In some embodiments, the first expansion is performed using a gas permeable container. In some embodiments, the second expansion is performed using a gas permeable container. In some embodiments, the ratio of the second population of TILs to the population of aAPCs in the rapid expansion is between 1 to 80 and 1 to 400. In some embodiments, the ratio of the second population of TILs to the population of aAPCs in the rapid expansion is about 1 to 300.

A further aspect relates to a population of tumor infiltrating lymphocytes (TILs) obtainable by a method comprising: culturing autologous T cells by obtaining a first population of TILs from a tumor resected from a mammal performing a first expansion by culturing the first population of TILs in a cell culture medium comprising IL-2 and one or more TME stimulators to produce a second population of TILs; and performing a second expansion by supplementing the cell culture medium of the second population of TILs with additional IL-2 and/or other cytokines from the “cytokine” group, anti-CD3, and antigen presenting cells (APCs), to produce a third population of TILs, wherein the third population of TILs is a therapeutic population.

A further aspect relates to a therapeutic population of TILs comprising IL-2 and one or more TME stimulators.

A further aspect relates to a therapeutic population of TILs comprising IL-2, one or more TME stimulators, IL-2, anti-CD3, and antigen presenting cells (APCs).

### Tables

TABLE 1
Flow cytometry antibodies

| Marker | clone | company |
|---|---|---|
| CD3 | UCHT1 | BD Biosciences |
| CD4 | SK3 | BD Biosciences |
| CD8 | RPA-T8 | BD Biosciences |
| IFN-g | B27 | BD Biosciences |
| TNF-a | Mab11 | BD Biosciences |
| CD107a | H4A3 | BD Biosciences |
| FVS780 | | BD Biosciences |
| CD56 | B159 | BD Biosciences |
| CCR7 | 2-L1-A | BD Biosciences |
| CD45RA | HI100 | BD Biosciences |
| LAG-3 | T47-530 | BD Biosciences |
| PD-1 | MIH4 | BD Biosciences |
| TIM-3 | 7D3 | BD Biosciences |
| BTLA | J168-540 | BD Biosciences |
| CD57 | NK-1 | BD Biosciences |
| CD28 | CD28.2 | BD Biosciences |
| CD27 | L128 | BD Biosciences |
| CD69 | FN50 | BD Biosciences |
| CD39 | TU66 | BD Biosciences |
| Dynabeads | | Life |
| Human T- | | technologies |
| Activator | | |
| CD3/CD28/CD137 | | |
| Protein transport | | BD Biosciences |
| inhibitor, Golgi | | |
| Plug | | |
| Foxp3/ | | Ebioscience |
| Transcription | | |
| Factor Staining | | |
| Buffer Set | | |
| Brilliant Stain | | BD Biosciences |
| buffer | | |

TABLE 2

| | Target | Name | Manufacturer |
|---|---|---|---|
| Group A | PD-1 | Pembrolizumab | Merck |
| Group B | CTLA-4 | Ipilimumab | Bristol-Meyers Squibb |
| Group C | LAG-3 | Relatlimab | Creative Biolabs |
| | | Biosimilar | |
| Group D | TIGIT | Tiragolumab | Creative Biolabs |
| | | Biosimular | |
| Group J | 4-1BB (CD137) | Urelumab | Creative Biolabs |
| | | Biosimular | |

TABLE 3
“Inhibitors” by antagonizing/inhibiting receptors
expressed on T-cells (or their ligands) known to cause
T-cell downregulation/deactivation/exhaustion:

| Group | Receptor on T-cell | antagonist examples |
|---|---|---|
| A | PD-1 | Pembrolizumab |
| A | PD-1 | Nivolumab |
| A | PD-1 | Cemiplimab |
| A | PD-1 | Sym021 |
| A | PD-1 | Toripalimab |
| A | PD-1 | Sintilimab |
| A | PD-1 | Camrelizumab |
| A | PD-1 | Tislelizumab |
| A | PD-1 | Sasanlimab |
| A | PD-1 | Dostarlimab |
| A | PD-L1 | Durvalumab |
| A | PD-L1 | Avelumab |
| A | PD-L1 | Atezolizumab |
| A (SMI) | PD-1 inhibitor | MAX-10181 |
| A (SMI) | PD-1/PD-L1 interaction | YPD-29B |
| A (SMI) | PD-1/PD-L1 interaction | IMMH-010 |
| A (SMI) | PD-L1 inhibitor | INCB086550 |
| A (SMI) | PD-1/PD-L1 interaction | DPPA-1 |
| A (SMI) | PD-1/PD-L1 interaction | TPP-1 |
| A (SMI) | PD-1/PD-L1 interaction | BMS-202 |
| A (SMI) | PD-1/PD-L1 interaction | CA-170 |
| A (SMI) | PD-L1 expression | JQ1 |
| A (SMI) | PD-L1 expression | eFT508 |
| A (SMI) | PD-L1 expression | Osimertinib |
| A (SMI) | PD-L1 expression | PlatycodinD |
| A (SMI) | PD-L1 degradation | PD-LYLSO |
| A (SMI) | PD-L1 degradation | Curcumin |
| A (SMI) | PD-L1 degradation | Metformin |
| B | | |
| B | CTLA-4 | Tremelimumab |
| C | LAG-3 | Relatlimab |
| C | | |
| C | LAG-3 | Sym022 |
| C(SMI) | LAG-3 | BMS-986016 |
| C(SMI) | LAG-3 | GSK28-31781 |
| D | TIGIT/CD226 | Tiragolumab |
| D(SMI) | | Liothyronine |
| E | KIR | Lirilumab |
| F | TIM-3 | Sym023 |
| G | BTLA | 40E4 and PJ196 |
| H | | A2aR |
| “Stimulators” by agonizing/stimulating | | |
| receptors expressed on T-cells known to | | |
| cause T-cell | | |
| upregulation/activation/reinvigoration: | | |
| Group | Receptor on T-cell | agonist examples |
| I | OX40/CD134 | |
| J | 4-1BB | Urelumab |
| J | 4-1BB | Utomilumab |
| J (Peptide) | | |
| J (Peptide) | 4-1BB | BCY7838 |
| K | CD28 | Theraluzimab |
| L | ICOS | |
| M | GITR | |
| N | CD40L | |
| O | CD27 | Varlilumab |
| W | CD3 | OKT-3 |
| W | CD3 | UCHT1 |
| W | CD3 | HIT3a |
| “Inhibitors” by antagonizing/inhibiting | | |
| soluble molecules and cytokines and their | | |
| receptors known to cause T-cell | | |
| downregulation/deactivation/exhaustion: | PD-1 | Dostarlimab |
| | Soluble | |
| Group | factor/cytokine | antagonist examples |
| P | IDO1/2 | Epacadostat |
| P | IDO1/2 | Linrodostat |
| Q | TGFβ | Galunisertib |
| R | IL-10 | |
| S | IL-35 | |
| “Inhibitors” by adding factors know to | | |
| downregulate and/or deplete regulator T- | | |
| cells thereby favoring ex-vivo effector T- | | |
| cell expansion: | PD-L1 inhibitor | GS-4224 |
| | Soluble | |
| Group | factor/inhibitor | |
| T | cyclophosphamide | |
| U | TKIs | sunitinib |
| U | TKIs | sorafenib |
| U | TKIs | imatinib |
| V | αCD25 | Daclizumab |
| | IL2/Diphteria toxin | |
| X | fusion | Dinileukin Diftitox |
| “Cytokines” | | PD-LYLSO |
| Group | | |
| Z1 | IL-2 | |
| Z2 | IL-7 | |
| Z3 | IL-12 | |
| Z4 | IL-15 | |
| Z5 | IL-21 | |
| C | LAG-3 | Relatlimab |
| C | LAG-3 | Eftilagimo alpha |
| C | LAG-3 | Sym022 |
| C(SMI) | LAG-3 | BMS-986016 |
| C(SMI) | LAG-3 | GSK28-31781 |
| D | TIGIT/CD226 | Tiragolumab |
| D(SMI) | | Liothyronine |
| E | KIR | Lirilumab |
| F | TIM-3 | Sym023 |
| G | BTLA | 40E4 and PJ196 |
| H | A2aR | |

“Stimulators” by agonizing/stimulating receptors expressed on T-cells
known to cause T-cell upregulation/activation/reinvigoration:

| Group | Receptor on T-cell | agonist examples |
|---|---|---|
| I | OX40/CD134 | |
| J | 4-1BB | Urelumab |
| J | 4-1BB | Utomilumab |
| J (Peptide) | 4-1BB | BCY7835 |
| J (Peptide) | 4-1BB | BCY7838 |
| K | CD28 | Theraluzimab |
| L | ICOS | |
| M | GITR | |
| N | CD40L | |
| O | CD27 | Varlilumab |
| W | CD3 | OKT-3 |
| W | CD3 | UCHT1 |
| W | CD3 | HIT3a |

“Inhibitors” by antagonizing/inhibiting soluble molecules and cytokines and their
receptors known to cause T-cell downregulation/deactivation/exhaustion:

| | Soluble | |
|---|---|---|
| Group | factor/cytokine | antagonist examples |
| P | IDO1/2 | Epacadostat |
| P | IDO1/2 | Linrodostat |
| Q | TGFβ | Galunisertib |
| R | IL-10 | |
| S | IL-35 | |

“Inhibitors” by adding factors know to downregulate and/or deplete
regulator T-cells thereby favoring ex-vivo effector T-cell expansion:

| | Soluble | |
|---|---|---|
| Group | factor/inhibitor | |
| T | cyclophosphamide | |
| U | TKIs | sunitinib |
| U | TKIs | sorafenib |
| U | TKIs | imatinib |
| V | αCD25 | Daclizumab |
| | IL2/Diphteria toxin | |
| X | fusion | Dinileukin Diftitox |
| “Cytokines” | | |
| Group | | |
| Z1 | IL-2 | |
| Z2 | IL-7 | |
| Z3 | IL-12 | |
| Z4 | IL-15 | |
| Z5 | IL-21 | |

TABLE 4
Reagents, antibodies and streptavidin
conjugates for tetramer staining

| Reagent | Number, clone | company |
|---|---|---|
| CD3-FITC | 561807, UCHT1 | BD Biosciences |
| CD8-BV480 | 566121, RPA-T8 | BD Biosciences |
| Dasatinib | #D-3307 | LC Laboratories |
| NIR live/dead | #15550745 | FisherScientific |
| HLA-A0201 | | DTU-produced |
| monomer | | |
| Top30 Peptides | | Pepscan, custom order |
| APC-SA | #405207 | Biolegend |
| PE-SA | #405204 | Biolegend |
| PerCP-ef710-SA | #46-4317-82 | FisherScientific |
| PE-CF594-SA | #562284 | BD Biosciences |
| PE-Cy7-SA | #557598 | BD Biosciences |
| APC-R700-SA | #565144 | BD Biosciences |
| BUV737-SA | #612775 | BD Biosciences |
| BV786-SA | #563585 | BD Biosciences |
| BV650-SA | #564876 | BD Biosciences |
| Brilliant Stain | #566349 | BD Biosciences |
| buffer | | |

TABLE 5
Peptide sequences and their origin used in tetramer screen

| No. | HLA | Protein | Sequence | cancer antigen class |
|---|---|---|---|---|
| 1 | A2 | MAGE-A2 | LVHFLLLKY | Cancer/Testis |
| 2 | A2 | gp100/Pmel17 | IMDQVPFSV | Melanoma Differentiation |
| 3 | A2 | CDKN1A | GLGLPKLYL | Overexpressed |
| 4 | A2 | gp100/Pmel17 | YLEPGPVTA | Melanoma Differentiation |
| 5 | A2 | Melan-A/MART-1 (WT) | EAAGIGILTV | Melanoma Differentiation |
| 6 | A2 | MAGE-C2 | KVLEFLAKL | Cancer/Testis |
| 7 | A2 | MAGE-A10 | SLLKFLAKV | Cancer/Testis |
| 8 | A2 | gp100/Pmel17 | KTWGQYWQV | Melanoma Differentiation |
| 9 | A2 | STEAP1 | MIAVFLPIV | Overexpressed |
| 10 | A2 | Telomerase | RLFFYRKSV | Overexpressed |
| 11 | A2 | LAGE-1 | MLMAQEALAFL | Cancer/Testis |
| 12 | A2 | MAGE-A2 | YLQLVFGIEV | Cancer/Testis |
| 13 | A2 | MAGE-C2 | LLFGLALIEV | Cancer/Testis |
| 14 | A2 | STAT1-alpha/β | KLQELNYNL | Overexpressed |
| 15 | A2 | TAG-1 | SLGWLFLLL | Cancer/Testis |
| 16 | A2 | MC1R | TILLGIFFL | Melanoma Differentiation |
| 17 | A2 | Melan-A/MART-1 | ILTVILGVL | Melanoma Differentiation |
| 18 | A2 | TRP-2 | FVWLHYYSV | Melanoma Differentiation |
| 19 | A2 | KIF20A | AQPDTAPLPV | Overexpressed |
| 20 | A2 | MAGE-A10 | GLYDGMEHL | Cancer/Testis |
| 21 | A2 | p53 | RMPEAAPPV | Overexpressed |
| 22 | A2 | SSX-2 | KASEKIFYV | Cancer/Testis |
| 23 | A2 | STEAP1 | FLYTLLREV | Overexpressed |
| 24 | A2 | TRP-2 | SVYDFFVWL | Melanoma Differentiation |
| 25 | A2 | GnTV | VLPDVFIRCV | Overexpressed |
| 26 | A2 | Livin (ML-IAP) | SLGSPVLGL | Overexpressed |
| 27 | A2 | MAGE-A1 | YLEYRQVPV | Cancer/Testis |
| 28 | A2 | Meloe-1 | TLNDECWPA | Overexpressed |
| 29 | A2 | NY-ESO-1/LAGE-2 | SLLMWITQC | Cancer/Testis |
| 30 | A2 | TRAG-3 | ILLRDAGLV | Cancer/Testis |

TABLE 6
Patient list

| Name | Diagnosis | Gender | Age | Ethnicity | Pathological Description | Stage | Prior Treatment |
|---|---|---|---|---|---|---|---|
| MM1 | Melanoma | Female | 76 | Caucasian | Nodular melanoma | II | treatment naïve |
| MM2 | Melanoma | Female | 48 | Caucasian | Malignant melanoma, NOS | IIIB | treatment naïve |
| MM3 | Melanoma | Male | 56 | Caucasian | Spindle cell melanoma, NOS | IIB | treatment naïve |
| HN1 | Head and neck | Male | 64 | Caucasian | Squamous cell carcinoma, NOS | III | treatment naïve |
| | cancer | | | | | | |
| HN2 | Head and neck | Male | 69 | Caucasian | Squamous cell carcinoma, NOS | III | treatment naïve |
| | cancer | | | | | | |
| HN3 | Head and neck | Male | 67 | Caucasian | Squamous cell carcinoma, NOS | IV | treatment naïve |
| | cancer | | | | | | |
| CC1 | Colorectal | Female | 88 | Caucasian | Mucinous adenocarcinoma | IIA | treatment naïve |
| | carcinoma | | | | | | |
| CC2 | Colorectal | Male | 81 | Caucasian | Adenocarcinoma, NOS | I | treatment naïve |
| | carcinoma | | | | | | |
| OC1 | Ovarian carcinoma | Female | 56 | Caucasian | Adenocarcinoma, NOS | IIIC | treatment naïve |
| OC2 | Ovarian carcinoma | Female | 52 | Caucasian | Adenocarcinoma, NOS | IV | treatment naïve |
| OC3 | Ovarian carcinoma | Female | 54 | Caucasian | Serous adenocarcinoma | IIIA | treatment naïve |
| LC1 | NSCLC | Male | 72 | Caucasian | Squamous cell carcinoma, NOS | IB | treatment naïve |
| LC2 | NSCLC | Male | 62 | Caucasian | Squamous cell carcinoma, NOS | IB | treatment naïve |
| Ce1 | Cervical cancer | Female | 64 | Caucasian | Squamous cell carcinoma, NOS | IB | treatment naïve |
| Ce2 | Cervical cancer | Female | 59 | Caucasian | Squamous cell carcinoma, NOS | IB | treatment naïve |
| Ce3 | Cervical cancer | Female | 47 | Caucasian | Squamous cell carcinoma, NOS | IIIB | treatment naïve |
| Ce4 | Cervical cancer | Female | 62 | Caucasian | Squamous cell carcinoma, NOS | IIB | treatment naïve |
| Ce5 | Cervical cancer | Female | 46 | Caucasian | Squamous cell carcinoma, NOS | IB | treatment naïve |
| NY-01 | Kidney cancer | Female | 70 | N/A | Clear cell renal cell carcinoma | N/A | treatment naïve |
| NY-02 | Kidney cancer | Male | 77 | N/A | Clear cell renal cell carcinoma | N/A | treatment naïve |
| NY-03 | Kidney cancer | Male | 64 | N/A | Clear cell renal cell carcinoma | N/A | treatment naïve |
| NY-04 | Kidney cancer | Female | 59 | N/A | Clear cell renal cell carcinoma | N/A | Trastuzumab for |
| | | | | | | | mammacancer |
| NY-05 | Kidney cancer | Male | 81 | N/A | Clear cell renal cell carcinoma | N/A | treatment naïve |
| NY-06 | Kidney cancer | Male | 57 | N/A | Clear cell renal cell carcinoma | N/A | treatment naïve |
| NY-08 | Kidney cancer | Male | 59 | N/A | Unclassified renal cell carcinoma | N/A | Axitinib 32 series until |
| | | | | | | | April 2021 |
| NY-11 | Kidney cancer | Female | 77 | N/A | Clear cell renal cell carcinoma | N/A | treatment naïve |
| NY-12 | Kidney cancer | Male | 50 | N/A | Clear cell renal cell carcinoma | N/A | treatment naïve |
| NY-13 | Kidney cancer | Male | 51 | N/A | Clear cell renal cell carcinoma | N/A | treatment naïve |
| OvCa- | Ovarian cancer | Female | 72 | N/A | High grade serous carcinoma | N/A | treatment naïve |
| 01 | | | | | | | |
| OvCa- | Ovarian cancer | Female | 76 | N/A | High grade serous carcinoma | N/A | treatment naïve |
| 02 | | | | | | | |
| OvCa- | Ovarian cancer | Female | 74 | N/A | High grade serous carcinoma | N/A | treatment naïve |
| 04 | | | | | | | |
| OvCa- | Ovarian cancer | Female | 45 | N/A | High grade serous carcinoma | N/A | treatment naïve |
| 06 | | | | | | | |
| OvCa- | Ovarian cancer | Female | 62 | N/A | High grade serous carcinoma | N/A | treatment naïve |
| 07 | | | | | | | |
| OvCa- | Ovarian cancer | Female | 78 | N/A | High grade serous carcinoma | N/A | treatment naïve |
| 08 | | | | | | | |
| OvCa- | Ovarian cancer | Female | 59 | N/A | High grade serous carcinoma | N/A | treatment naïve |
| 09 | | | | | | | |
| LC3 | Lung cancer | Female | 48 | N/A | Adenocarcinoma | I | N/A |
| LC4 | Lung cancer | Male | 77 | N/A | Pulmonary adenocarcinoma | IA3 | N/A |
| LC5 | Lung cancer | Male | 65 | N/A | Non-small cell lung cancer | IIIB | N/A |

### General

It should be understood that any feature and/or aspect discussed above in connections with the compounds according to the invention apply by analogy to the methods described herein.

The following figures and examples are provided below to illustrate the present invention. They are intended to be illustrative and are not to be construed as limiting in any way.

### BRIEF DESCRIPTION OF THE FIGURES

FIG. 1 shows frequency of CD8+ and CD4+ T cells expressing for all cancer types using IL-2+/− TME-S. Shown is the median +95% Cl interval. Refer to table 2 for the specific stimulator of each group.

FIG. 2 shows total number of viable CD8+ T cells per fragment for all cancer types using IL-2+/− TME-S. Shown is the median +95% Cl interval. Refer to table 2 for the specific stimulator of each group. Statistics performed by two-tailed Mann-Whitney U test. p>0.05 was considered non-significant. *p<0.05, **p<0.01, ***p<0.001, ****p<0.0001.

FIG. 3 shows total number of CD8+ T cells per fragment expressing either one, two or three of the cytotoxic degranulation marker CD107a, or the cytokines IFNg and TNFa for cervical cancer using IL-2+/− TME-S. Shown is the median +95% Cl interval. Refer to table 2 for the specific stimulator of each group.

FIG. 4 shows total number of CD8+ T cells per fragment expressing either one, two or three of the cytotoxic degranulation marker CD107a, or the cytokines IFNg and TNFa for cervical cancer using IL-2+/− TME-S. Shown is the median +95% Cl interval. Refer to table 2 for the specific stimulator of each group.

FIG. 5 shows total number of CD8+ T cells per fragment expressing either one, two or three of the cytotoxic degranulation marker CD107a, or the cytokines IFNg and TNFa for cervical cancer using IL-2+/− TME-S. Shown is the median +95% Cl interval. Refer to table 2 for the specific stimulator of each group. Statistics performed by two-tailed Mann-Whitney U test. p>0.05 was considered non-significant. The numbers on the figure are the calculated p values.

FIG. 6 shows time in culture and success rate for TIL cultures. TIL cultures were established with renal cell carcinoma, ovarian cancer, cervical cancer and lung cancer fragments. The cultures were maintained and harvested as described in Example 6. Figure A shows the time from culture start plotted against the number of viable cells per fragments at harvest. The dotted line represents 100.000 viable cells per fragment. The minimum required cells for a clinical scale TIL product. Figure B shows the success rate for the different conditions defined as an expansion to more than 100.000 viable cells per fragment.

FIG. 7 shows T and NK cells and T cell subsets in TIL cultures with TME stimulators. TIL cultures were established with kidney, ovarian, cervical and lung cancer fragments. Cultures with indicated conditions were established. Scatter plots showing (A) % T and NK cells of live (B) % CD4 and CD8 cells of T cells. Data are presented as median with 95% Cl. *P<0.05, **P<0.01 by 2way ANOVA.

FIG. 8 shows T cell expansion in cultures treated with TME stimulators. TIL cultures were established with kidney, ovarian, cervical and lung cancer fragments. TIL cultures with indicated conditions were established. Scatter plots showing viable cells per fragment for (A) CD3+ T cells (B) CD8+ T cells (C) CD4+ T cells. Data are presented as median with 95% Cl. *P<0.05, **P<0.01, ***P<0.001 by Mann-Whitney test.

FIG. 9 shows expression of activation markers by CD8 T cells from TIL cultures with TME stimulators. TIL cultures were established with kidney, ovarian, cervical and lung cancer fragments. Cultures with indicated conditions were established. Scatter plots showing % of (A) BTLA+(B) LAG3+(C) TIM3+(D) CD28+(E) CD28+(F) CD57+ CD8 T cells. Data are presented as median with 95% Cl. *P<0.05, **P<0.01 by Mann-Whitney test.

FIG. 10 shows CD8 T cell differentiation in TIL cultures with TME stimulators. TIL cultures were established with kidney, ovarian, cervical and lung cancer fragments. Cultures with indicated conditions were established. Summary of CD8 T cell subsets Tem (CCR7-CD45RA−), Temra (CCR7−, CD45RA+), Tcm (CCR7+, CD45RA−), Tnaive (CCR7+, CD45RA+). Data are presented as median with 95% Cl. *P<0.05 by 2way ANOVA.

FIG. 11 shows stem-like CD8 T cells in TIL cultures with TME stimulators. TIL cultures were established with kidney, ovarian, cervical and lung cancer fragments. Cultures with indicated conditions were established. (A) Summary of CD8 T cell subsets based on CD39 and CD69 expression. Scatter plots showing % of (B) CD39+ CD69+ and (C) CD39− CD69− CD8 T cells. (D) Scatter plot showing the number of stem-like (CD39− CD69−) CD8 T cells in the cultures. Data are presented as median with 95% Cl. *P<0.05, **P<0.01, ***P<0.001, ****P<0.0001 by Mann-Whitney test.

FIG. 12 shows a summary of the effect of time delay (TD) compared to non time delay (non TD) in TIL cultures with TME stimulators. TIL cultures were established with kidney, ovarian, cervical and lung cancer fragments. Data from JAB and JAB+C+D conditions were pooled as non TD and data from JAB TD and JAB+C+D TD conditions were pooled as TD. Scatter plots showing (A) viable CD3+ T cells per fragment (B) viable CD8+ T cells per fragment (C) % of TIM3+ CD8 T cells (D) % of CD28+ CD8 T cells (E) % of CD39+ CD69+ CD8 T cells (F) % of CD39− CD69− CD8 T cells. Data are presented as median with 95% Cl. *P<0.05, **P<0.01, ***P<0.001, ****P<0.0001 by Mann-Whitney test.

FIG. 13 shows a higher frequency of CD8 T cells with high cytotoxic capacity in TIL cultures with TME stimulators. TIL cultures were established with kidney and ovarian cancer fragments with indicated conditions and young TILs stimulated with dynabeads coated with αCD3, αCD28 and a4− 1BB and then stained for reactivity markers IFNγ, TNFα and CD107a. (A) Proportion of CD8+ T cells either single-, double- or triple positive for all combinations of IFNγ, TNFα and CD107a. Total % indicates the total fraction of reactive CD8+ cells.

FIG. 14 shows CD8 T cells with high cytotoxic capacity in TIL cultures with TME stimulators. TIL cultures were established with kidney and ovarian cancer fragments with indicated conditions and young TILs stimulated with dynabeads coated with αCD3, αCD28 and a4-1BB and then stained for reactivity markers IFNγ, TNFα and CD107a. (A) Scatter plot showing the total number of reactive CD8+ T cells in the different cultures. Data are presented as median with 95% Cl. (B) Scatter plot showing the number of triple-positive CD8+ T cells in the different cultures. Data are presented as median with 95% Cl. *P<0.05, **P<0.01, ***P<0.001, ****P<0.0001 by Mann-Whitney test.

FIG. 15 shows CD8 T cells with high cytotoxic capacity in TIL cultures with TME stimulators with Time Delay. TIL cultures were established with kidney and ovarian cancer fragments with indicated conditions and young TILs stimulated with dynabeads coated with αCD3, αCD28 and a4-1BB and then stained for reactivity markers IFNγ, TNFα and CD107a. (A) Scatter plot showing the number of reactive CD8+ T cells in the different cultures. Data are presented as median with 95% Cl. (B) Scatter plot showing the number of triple-positive CD8+ T cells in the different cultures. Data are presented as median with 95% Cl. *P<0.05, **P<0.01, ***P<0.001, ****P<0.0001 by Mann-Whitney test.

FIG. 16 shows time delay increases the proportion of reactive T-cells for some patient samples TIL cultures were established with kidney and ovarian cancer fragments as described in example 7. Cultures with indicated conditions were established and young TILs stimulated with dynabeads coated with αCD3, αCD28 and a4-1BB and then stained for reactivity markers IFNγ, TNFα and CD107a. The proportion of reactive cells (single-, double- or triple positive for combinations of IFNγ, TNFα and CD107a) were plotted against the number of viable CD8+ cells obtained in each culture. Closed figures represent cell cultures from OV7 and open figures samples from patient OV9. Square represents the IL-2 cultures. Circle cultures stimulated with JAB or JAB+C+D and triangles represents cultures stimulated with JAB or JAB+C+D with a time delay. *P<0.05, **P<0.01, ***P<0.001, ****P<0.0001 by Mann-Whitney test.

FIG. 17 shows detected CD8+ T cell populations specific for cancer-associated antigens with pMHC tetramers. TIL cultures were established with cervical cancer fragments. Cultures with indicated conditions were established and young TILs stained with a library of 30 cancer/testis pMHC tetramers to identify T cell specificities. (A) List of identified specific CD8+ T cells populations across the three Cervical cancer patient samples Ce1, Ce3 and Ce4. Cultures were grown from five tumor fragments, unless otherwise indicated. The number indicates the % of tetramer-positive cells within the CD8+ population.

FIG. 18 shows detected CD8+ T cell populations specific for cancer-associated antigens with pMHC tetramer across cervical cancer patients. TIL cultures were established with cervical cancer fragments. Cultures with indicated conditions were established and young TILs stained with a library of 30 cancer/testis pMHC tetramers to identify T cell specificities. (A)+(B) Cultures were grown from five tumor fragments, unless otherwise indicated. Plot showing the number and sum of frequencies of reactive CD8+ T cell populations in the different cultures. Each line represents one Cervical cancer patient. Triangles represent samples made from ten tumor fragments instead of five.

FIG. 19 shows number and frequency of detected CD8+ T cell populations specific for cancer-associated antigens are higher in TD samples than in non TD samples. TIL cultures were established with cervical cancer fragments. Cultures with indicated conditions were established and young TILs stained with a library of 30 cancer/testis pMHC tetramers to identify T cell specificities. (A)+(B) Plot showing the number and sum of frequencies of reactive CD8+ T cell populations in the different cultures.

FIG. 20 shows viable cells per fragment for TIL cultures cultured in IL2, JAB, JAB TD (48 h) and JAB TD (96 h) TIL cultures were established with kidney, ovarian and cervical cancer fragments (1 Ce, 2 kidney and 3 Ovarian). The cultures were maintained and harvested as described in Example 7.

FIG. 21 shows T and NK cells and T cell subsets in TIL cultures with TME stimulators. TIL cultures were established with kidney, ovarian, cervical and lung cancer fragments. Cultures with indicated conditions were established. Scatter plots showing (A) % T and NK cells of live (B) % CD4 and CD8 cells of T cells. Data are presented as median with 95% Cl. ****P<0.0001 by 2way ANOVA.

FIG. 22 shows T cell expansion in cultures treated with TME stimulators. TIL cultures were established with kidney, ovarian, cervical and lung cancer fragments. TIL cultures with indicated conditions were established. Scatter plots showing viable cells per fragment for (A) CD3+ T cells (B) CD8+ T cells (C) CD4+ T cells. Data are presented as median with 95% Cl. *P<0.05 by Mann-Whitney test.

FIG. 23 shows expression of activation markers by CD8 T cells from TIL cultures with TME stimulators. TIL cultures were established with kidney, ovarian, cervical and lung cancer fragments.

Cultures with indicated conditions were established. Scatter plots showing % of (A) BTLA+(B) LAG3+(C) TIM3+(D) CD28+(E) CD28+(F) CD57+ CD8 T cells. Data are presented as median with 95% Cl. *P<0.05, **P<0.01 by Mann-Whitney test.

FIG. 24 shows CD8 T cell differentiation in TIL cultures with TME stimulators. TIL cultures were established with kidney, ovarian, cervical and lung cancer fragments. Cultures with indicated conditions were established. Summary of CD8 T cell subsets Tem (CCR7− CD45RA−), Temra (CCR7−, CD45RA+), Tcm (CCR7+, CD45RA−), Tnaive (CCR7+, CD45RA+). Data are presented as median with 95% Cl. ****P<0.0001 by 2way ANOVA.

FIG. 25 shows stem-like CD8 T cells in TIL cultures with TME stimulators. TIL cultures were established with kidney, ovarian, cervical and lung cancer fragments. Cultures with indicated conditions were established. (A) Summary of CD8 T cell subsets based on CD39 and CD69 expression. Scatter plots showing % of (B) CD39+ CD69+ and (C) CD39− CD69− CD8 T cells. (D) Scatter plot showing the number of stem-like (CD39− CD69−) CD8 T cells in the cultures. Data are presented as median with 95% Cl. *P<0.05 by Mann-Whitney test.

### EXAMPLES

### Example 1—“Young” Tumor-Infiltrating Lymphocytes (TILs) with TME Stimulators

This example demonstrated the generation of “young” tumor-infiltrating lymphocytes (TILs) with TME stimulators.

Tumor material of various histologies were obtained from commercial sources. eighteen independent patient tumors or tumor digests were obtained (3 ovarian cancer, 3 metastatic melanoma, 3 head and neck cancer, 2 lung cancer, 2 colorectal cancer, 5 cervical cancer). The cervical cancer samples were shipped fresh in sterile transport media. The rest of the tumor samples were cryopreserved samples and were shipped to Cbio A/S in sterile freezing medium. The tumor material was handled in a laminar flow hood to maintain sterile conditions.

TILs were prepared as previously described in detail (Friese, C. et al., CTLA-4 blockade boosts the expansion of tumor-reactive CD8+ tumor-infiltrating lymphocytes in ovarian cancer. Sci Rep 10, 3914 (2020); Jin, J. et al., Simplified Method of the Growth of Human Tumor Infiltrating Lymphocytes in Gas-permeable Flasks to Numbers Needed for Patient Treatment, Journal of Immunotherapy, 35—Issue 3 (2012)). Briefly, TIL cultures were set up using tumor fragments or tumor digest. The tumors were divided into 1-3 mm³ fragments and placed into a G-Rex 6-well plate (WilsonWolf; 5 fragments per well) with 10 ml complete medium (CM) supplemented with 6000 IU/mL IL-2 (6000 IU/ml, Clinigen) only (baseline) or in combination with TME stimulators of each of the PD-1/PD-L1 antagonists (group A), CTLA-4 antagonist (group B), LAG-3 antagonist (group C), TIGIT antagonist (group D) and 4-1BB agonist (group J) in combination with anti-CD3, in a humidified 37° C. incubator with κ% CO₂ at the same time or with a time delay or time lapse. CM and IL-2 was added every 4-5 days until a total volume of 40 ml was reached. Subsequently, half of the medium was removed and replaced with CM and IL-2 every 4-5 days. TIL cultures from tumor digest were initiated by culturing single-cell suspensions (5×10⁵/ml) obtained by overnight enzymatic digestion in flat-bottom 96-well plates in 250 μL CM and IL-2 (6000 IU/ml, Clinigen) in a humidified 37° C. incubator with 5% CO₂. Half of the medium was removed and replaced with CM and IL-2 every 2-3 days.

CM consisted of RPM11640 with GlutaMAX, 25 mM HEPES pH 7.2 (Gibco), 10% heat-inactivated human AB serum (Sigma-Aldrich), 100 U/mL penicillin, 100 μg/mL streptomycin (Gibco), and 1.25 μg/ml Fungizone (Bristol-Myers Squibb).

This example demonstrated the generation of “young” tumor-infiltrating lymphocytes (TILs) with TME stimulators having an age of 10-28 days.

### Example 2—Phenotype Analysis of “Young” TIL Cultures with TME Stimulators

This example demonstrates the phenotype analysis of “young” TIL cultures with TME stimulators.

When cultures designated for young TIL generation were harvested, their phenotype was assessed by flow cytometry. TIL phenotype was determined by assessment of the viability and the CD3+ subset, the CD3+ CD8+ subset and the CD3+ CD4+ subset in both frequency and absolute cell count.

### TIL Panel: CD3, CD4, CD8, Live Dead Marker

Briefly, about 0.5×10⁶ young TILs were washed and then incubated with titrated antibodies (BD Biosciences, Table 1) and Brilliant Stain Buffer (BD Biosciences) for 30 min at 4° C. Cells were washed twice with PBS and directly analyzed by flow cytometry (CytoFLEX, Beckman Coulter).

This example demonstrated the phenotype analysis of “young” TIL cultures with TME stimulators.

### Example 3—TME-Stimulators in Combination Enhance the Frequency and Number of CD8+ T Cells and Reduce the Frequency of CD4+ T Cells

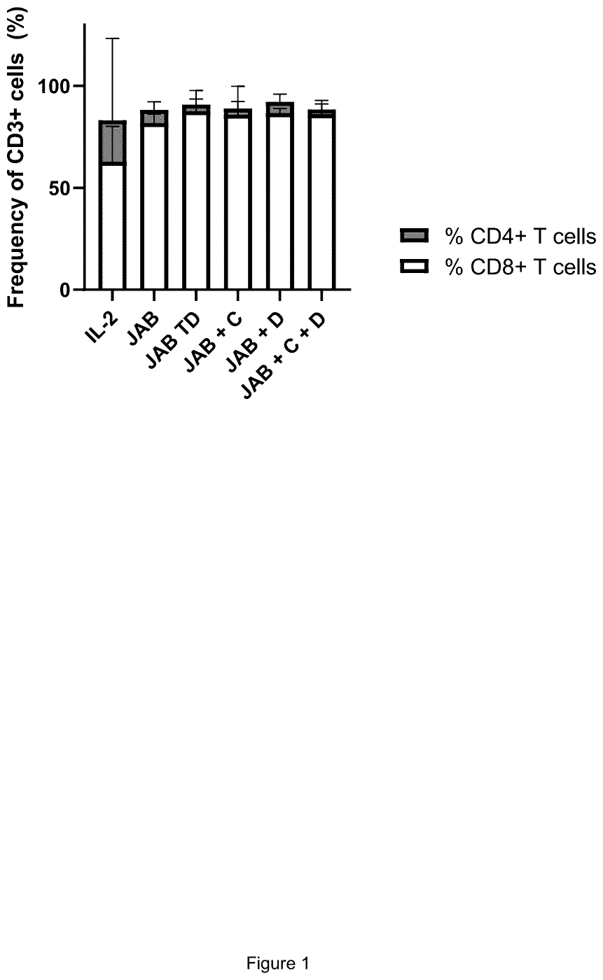

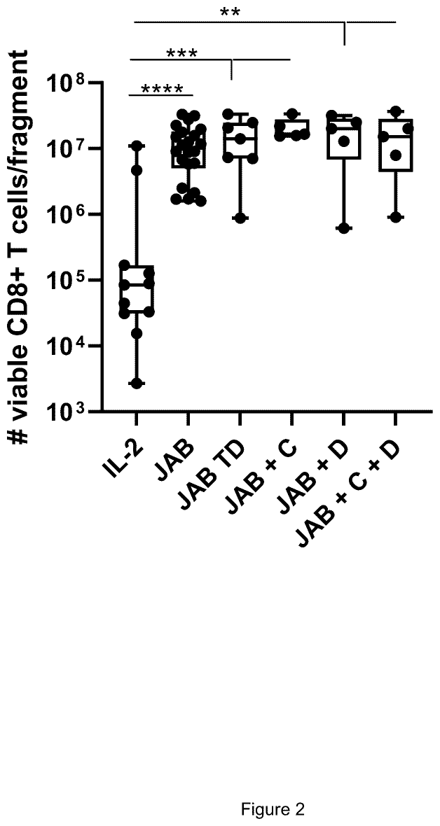

Example 3 illustrated in FIG. 1-2 demonstrated that adding a combination of TME stimulators from group J (4-1 BB inhibitors and ligand) in combination with anti-CD3, group A (including inhibitors of PD1 and its ligand PD-L1), group B (inhibitors of CTLA-4 and ligand), group C (LAG-3 antagonist) and group D (TIGIT antagonist) at the same time or group A (including inhibitors of PD1 and its ligand PD-L1), group B (inhibitors of CTLA-4 and ligand) on day 0 and group J (4-1 BB stimulators) in combination with anti-CD3 with a time delay or time lapse of 2 days to the standard young TIL protocol performed as described in example 1 and staining T cells using anti-CD3, anti-CD4 and anti-CD8 flow cytometry antibodies as described in example 2 significantly enhanced CD8+ T-cell growth which resulted in a significantly increased frequency (FIG. 1) and total number (FIG. 2) of CD8+ T cells compared to IL-2 alone and to a decreased frequency of CD4+ T cells (FIG. 1). This frequency of CD8+ T cells was even more increased when TME stimulator of group J in combination with anti-CD3 was added on day 2, compared to TME stimulators of group A, B and J in combination with anti-CD3 added at the same time.

This was illustrated using a representative number of tumor fragments from various solid cancers including ovarian, head and neck, colorectal, melanoma, cervical, colorectal, and lung cancer.

Summing up this example, adding TME stimulators with a time delay or time lapse to the young TIL processing step provided a novel improvement over the existing standard TIL protocol that allowed for generation of a TIL product containing an increased total number and frequency CD8+ T cells and a reduced frequency CD4+ T cells.

### Example 4—Cytotoxic Potential Analysis of “Young” TIL Cultures with TME Stimulators

This example demonstrates the analysis of the cytotoxic potential of “young” TIL cultures with TME stimulators performed as described in example 1.

When cultures designated for young TIL generation were harvested, their reactivity and cytotoxic potential was assessed by flow cytometry. Reactivity was assessed by stimulation of young TILs with CD3/CD28/CD137 coated beads and subsequent staining of cytotoxic degranulation marker CD107a on the cell surface and cytokines INFg and TNFa intracellularly. Characterization of T cell subsets was additionally analyzed using following markers:

TIL cell surface: CD107a, CD3, CD4, CD8

TIL intracellularly: INFg, TNFa

Briefly, about 2×10⁶ young TILs per sample were thawed and rested overnight in a 24-well plate in RPMI+ 10% inactivated human AB serum and 1% Pen/Strep. The next day, cells were harvested and counted. 1×10⁵ TILs were transferred to a 96 well plate in triplicates and stimulated with CD3/CD28/CD137 dynabeads with a bead-to-cell ratio of 1:10 for six hours in presence of aCD107a antibody and Golgi Plug.

After six hours, cells were washed and then incubated with titrated surface antibodies (BD Biosciences, Table 1) and PBS for 30 min at 4° C. Cells were washed twice with PBS+0.5% BSA and then fixed overnight at 4° C. with fixation buffer (FoxP3 Staining Buffer Set, ebioscience, Table 1). The next day cells were washed twice with Permeabilization buffer (FoxP3 Staining Buffer Set ebioscience, Table 1) and then stained for intracellular cytokine antibodies (BD biosciences, Table 1) and PBS for 30 min at 4° C. Cells were washed twice with Permeabilization buffer (FoxP3 Staining Buffer Set, ebioscience, Table 1), resuspended in PBS+0.5% BSA and directly analyzed by flow cytometry (CytoFLEX, Beckman Coulter).

This example demonstrates the reactivity and functionality analysis of “young” TIL cultures with TME stimulators.

### Example 5—TME-Stimulators in Combination Added with or without Time Delay or Time Lapse Enhance the Total Number of CD8+ T Cells with Cytotoxic Potential

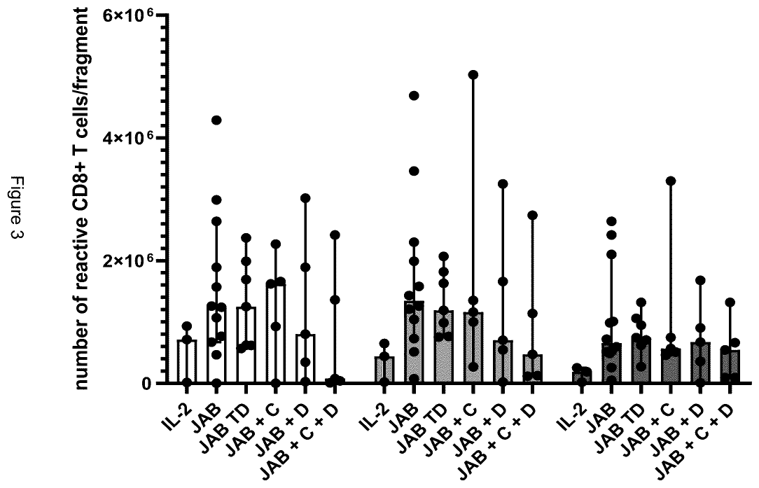

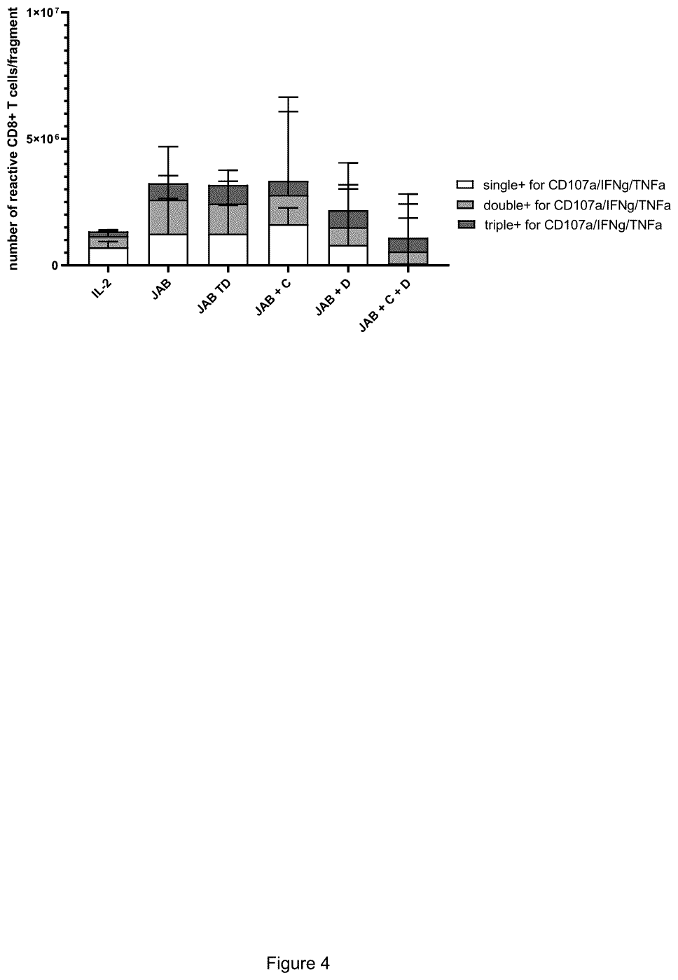

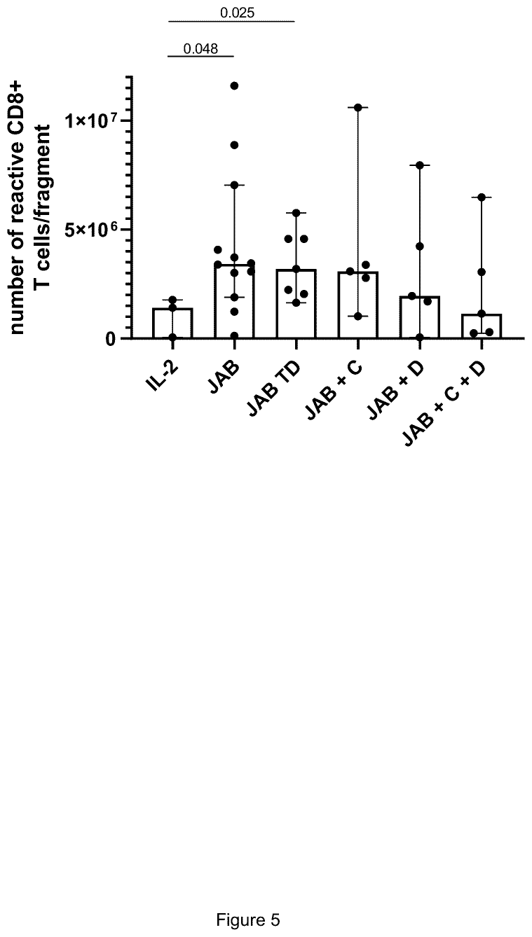

Example 5 illustrated in FIGS. 3, 4 and 5 demonstrated that adding a combination of TME stimulators from group J (4-1 BB stimulator) in combination with anti-CD3, group A (including inhibitors of PD1 and its ligand PD-L1), group B (inhibitors of CTLA-4 and ligand), group C (LAG-3 antagonist) and group D (TIGIT antagonist) at the same time or group A (including inhibitors of PD1 and its ligand PD-L1), group B (inhibitors of CTLA-4 and ligand) on day 0 and group J (4-1BB stimulators) in combination with anti-CD3 with a time delay or time lapse of 2 days to the standard young TIL protocol performed as described in example 1 and stimulating T cells using aCD3, aCD28 and a41BB coated beads followed by an (intra-)cellular staining with aCD107a, aCD3, aCD4, aCD8, alFNg and aTNFa as described in example 4 resulted in an increased total number of CD8+ T cells expressing either one, two or three of the cytotoxic markers (FIG. 3, 4) and resulted in a significantly higher number of reactive CD8+ T cells in total, compared to the IL-2 condition (FIG. 5), whereas adding the TME stimulators of group J in combination with anti-CD3, group A and group B with a time delay or time lapse led to a more significant increase of the total number of cytotoxic CD8+ T cells compared to adding the stimulators without time delay or time lapse (FIG. 5).

This was illustrated using a representative number of tumor fragments from cervical cancer. The combination of TME stimulators of group J in combination with anti-CD3, group A, group B, group C and group D with or without time delay or time lapse seems to increase the number of CD8 T cells with a cytotoxic potential compared to the standard protocol with IL-2.

### Example 6—“Young” Tumor-Infiltrating Lymphocytes (TILs) with TME Stimulators

This example demonstrated the generation of “young” tumor-infiltrating lymphocytes (TILs) with TME stimulators as described in Example 1 with following changes:

Tumor material of various histologies were obtained from commercial sources or collaborations with Odense University Hospital. 27 independent patient tumors (7 ovarian cancer, 10 renal cell carcinoma, 5 Cervical, 5 Lung Cancer, Table 6). Fresh tumor material was shipped to Cbio A/S in sterile transport media. The tumor material was handled in a laminar flow hood to maintain sterile conditions.

The tumors were divided into 1-3 mm³ fragments and placed into a G-Rex 6-well plate (WilsonWolf; 5 fragments per well unless otherwise indicated) with 5 ml complete medium (CM) supplemented with 6000 IU/mL IL-2 (6000 IU/ml, Clinigen) only (baseline) or in combination with TME stimulators of each of the PD-1/PD-L1 antagonists (group A), CTLA-4 antagonist (group B), LAG-3 antagonist (group C), TIGIT antagonist (group D) and 4-1 BB agonist (group J) in combination with anti-CD3, in a humidified 37° C. incubator with 5% CO₂ at the same time or with a time delay or time lapse of 2 days. TME stimulation combinations are called corresponding to the stimulator groups J, A, B, C, D, without or with time delay of 2 days (TD).

### Example 7—Culturing TILs with TME Stimulators Increases Cell Number and Success Rate while Reducing Culture Time

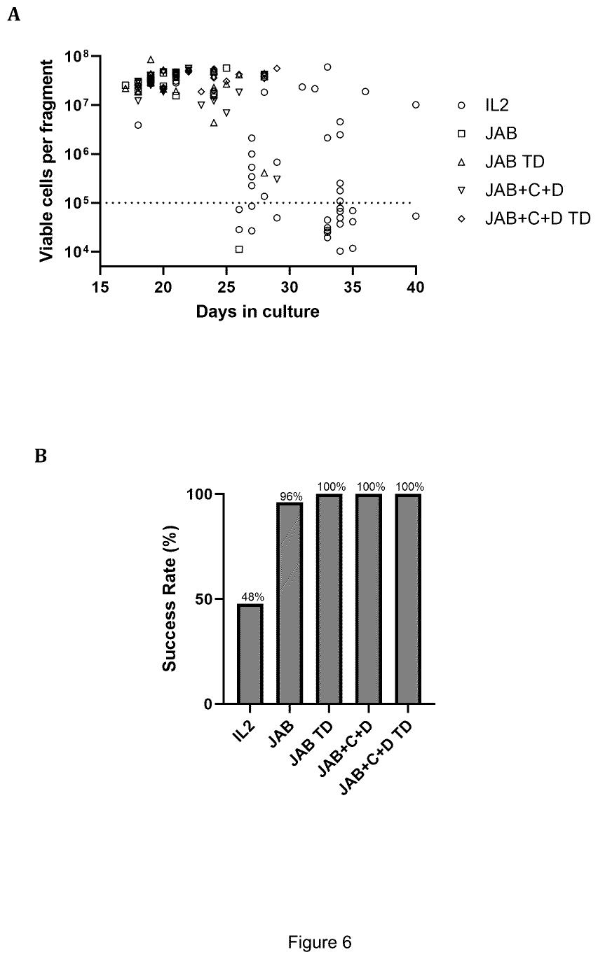

Example 7 illustrated in FIG. 6 demonstrated that adding a combination of TME stimulators as described in Example 6 to the standard young TIL protocol performed reduces days in culture to reach higher numbers of expanded TILs, as shown in FIG. 6 A. FIG. 6 B illustrates that adding the different combinations of TME stimulators significantly enhanced success rate of expanding young TILs from tumor fragments of ovarian cancer, renal cell carcinoma, cervical cancer and lung cancer compared to the standard IL-2 conditions from 48% to 96% for cultures with stimulators from group J (4-1 BB inhibitors and ligand), group A (including inhibitors of PD1 and its ligand PD-L1), group B (inhibitors of CTLA-4 and ligand) added at the same time (JAB) and to 100% for JAB TD, JAB+C+D and JAB+C+D TD.

This was illustrated using a representative number of tumor fragments from ovarian cancer, cervical cancer, lung cancer and renal cell carcinoma.

### Example 8—Phenotype Analysis of “Young” TIL Cultures with TME Stimulators

This example demonstrates the phenotype analysis of “young” TIL cultures with TME stimulators.

When cultures designated for young TIL generation as described in Example 6 were harvested, their phenotype was assessed by flow cytometry. TIL phenotype was determined by assessment of the viability and the CD3+ subset, the CD3−CD56+ subset, the CD3+CD8+ subset and the CD3+ CD4+ subset in both frequency and absolute cell count, and frequencies of CD8+ T cells expressing the phenotypic markers CD27, CD28, CD39, CD57, CD69, BTLA, LAG3, TIM3, CD45RA, CCR7.

TIL Panel: CD3, CD4, CD8, CD56, BTLA, LAG3, TIM3, CD28, CD27, CD57, CD39, CD69, CD45RA, CCR7, Live Dead Marker

Briefly, about 0.5×10⁶ young TILs were washed and then incubated with titrated antibodies (BD Biosciences, Table 1) and Brilliant Stain Buffer (BD Biosciences) for 30 min at 4° C. Cells were washed twice with PBS and directly analyzed by flow cytometry (CytoFLEX, Beckman Coulter).

This example demonstrated the phenotype analysis of “young” TIL cultures with TME stimulators of ovarian cancer, renal cell carcinoma, cervical and lung cancer fragments.

### Example 9—TME Stimulators in Combination Enhance the Frequency and Number of CD8+ T Cells

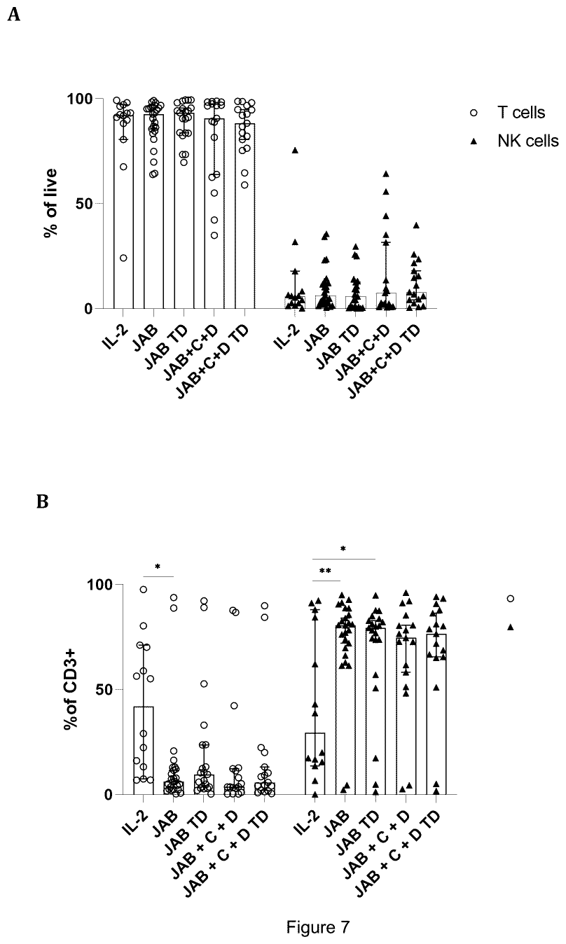

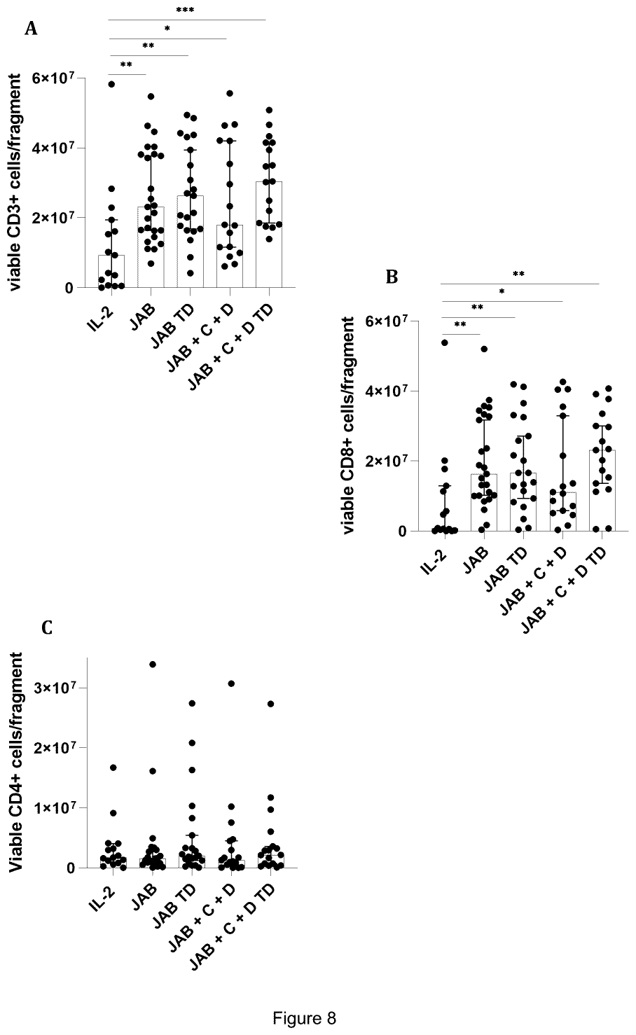

Example 9 illustrated in FIG. 7 and FIG. 8 demonstrated that adding a combination of TME stimulators as described in Example 6 to the standard young TIL protocol and staining T cells using anti-CD3, anti-CD4 and anti-CD8 flow cytometry antibodies as described in example 8 significantly enhanced T cell growth which resulted in similar frequencies of CD3 and NK cells (FIG. 7 A) and a significantly increased frequency of CD8+ T cells (FIG. 7 B) and total number of CD3+ (FIG. 8 A) and CD8+(FIG. 8 B) T cells in TME stimulator samples compared to IL-2 alone. No change in CD4+ T cell frequency or total number was detected in TME stimulator samples compared to IL-2. This was illustrated using a representative number of tumor fragments from ovarian cancer, cervical cancer, lung cancer and renal cell carcinoma.

Higher numbers of T cells, specifically CD8+ T cells, has been repeatedly shown to be associated with better outcome of adoptive TIL transfer (Radvanyi, 2012).

Summing up this example, adding TME stimulators without or with a time delay of 2 days to the young TIL processing step provided a novel improvement over the existing standard TIL protocol that allowed for generation of a TIL product containing an increased total number and frequency CD8+ T cells.

### Example 10—TME Stimulators in Combination Enhance the Frequency of BTLA+ and CD28+ CD8+ T Cells

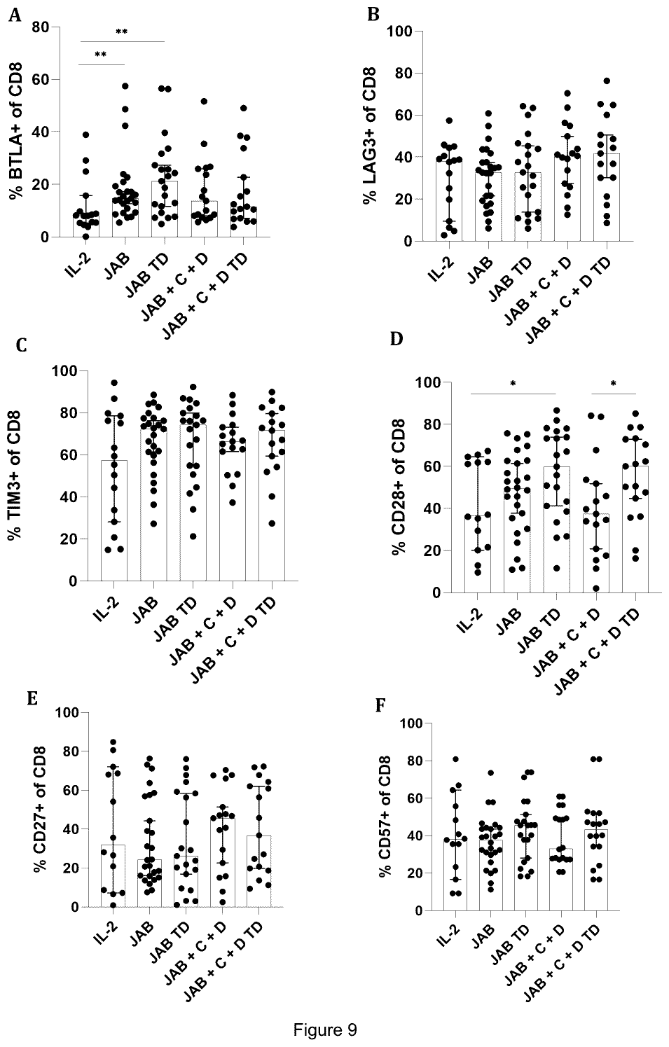

Example 10 illustrated in FIG. 9 demonstrated that adding a combination of TME stimulators as described in Example 6 to the standard young TIL protocol and staining T cells using anti-CD27, anti-CD28, anti-CD57, anti-BTLA, anti-LAG3 and anti-TIM3− flow cytometry antibodies as described in example 8 results in a significantly higher frequency of BTLA expressing CD8 T cells when adding TME stimulator combinations, with the tendency towards a higher percentage in JAB TD samples, as shown in FIG. 9A. Similarly, there was the tendency towards a higher frequency of TIM3+ CD8+ T cells in all samples grown with TME stimulators, but especially in JAB TD samples, as shown in FIG. 9 C.

Both markers have been described to be expressed on activated and cytotoxic CD8+ T cells, representing the tumor specific T cells fraction.

Additionally, a significantly higher frequency of CD28+ CD8+ T cells was detected in JAB TD and JAB+C+D TD samples (FIG. 9D), pointing towards a higher proportion of cells expressing the coactivation marker and therefore representing cells that are able to respond to stimulation and are capable of tumor recognition.

The other markers LAG3 (FIG. 9 B), CD27 (FIG. 9 E) and CD57 (FIG. 9 F) do not show large differences in the TME stimulator samples compared to the standard IL-2 condition or between each other.

This was illustrated using a representative number of tumor fragments from ovarian cancer, cervical cancer, lung cancer and renal cell carcinoma.

These results point towards an expansion of cells with a more activated and tumor-specific phenotype. Especially BTLA has been associated with better outcome of TIL infusion (Radvanyi, 2012). Expression of CD28 is mostly retained in TME stimulator expanded TILs or even significantly increased in JAB TD expanded TILs compared to the standard IL-2 condition, which points towards TILs that still express costimulatory molecules and can therefore be activated upon antigen recognition.

### Example 11—TME Stimulators in Combination Enhance the Frequency of CD8+ T Cells with an Effector Memory Phenotype

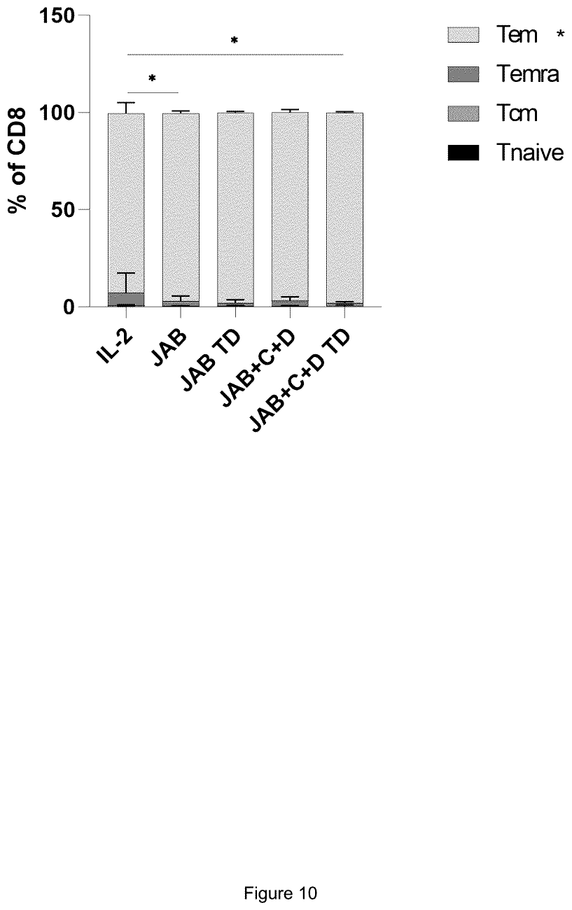

Example 11 illustrated in FIG. 10 demonstrated that adding a combination of TME stimulators as described in Example 6 to the standard young TIL protocol and staining T cells using anti-CD3, anti-CD8, anti-CD45RA and anti-CCR7 flow cytometry antibodies as described in example 8 resulted in an increase of CD8 TILs with an effector memory (CD54RA−, CCR7−) phenotype in TILs expanded with TME stimulators compared to the IL-2 conditions. Especially TILs expanded with JAB or JAB+C+D TD showed a significant increase in effector memory T cells.

This was illustrated using a representative number of tumor fragments from ovarian cancer, cervical cancer, lung cancer and renal cell carcinoma.

The effector-memory phenotype has repeatedly been associated with a favorable outcome of Adoptive Cell Therapy (ACT).

### Example 12—TME Stimulators in Combination Enhance the Frequency and Total Number of CD8+ T Cells that are Negative for CD39 and CD69 with a Stem-Cell Like Phenotype

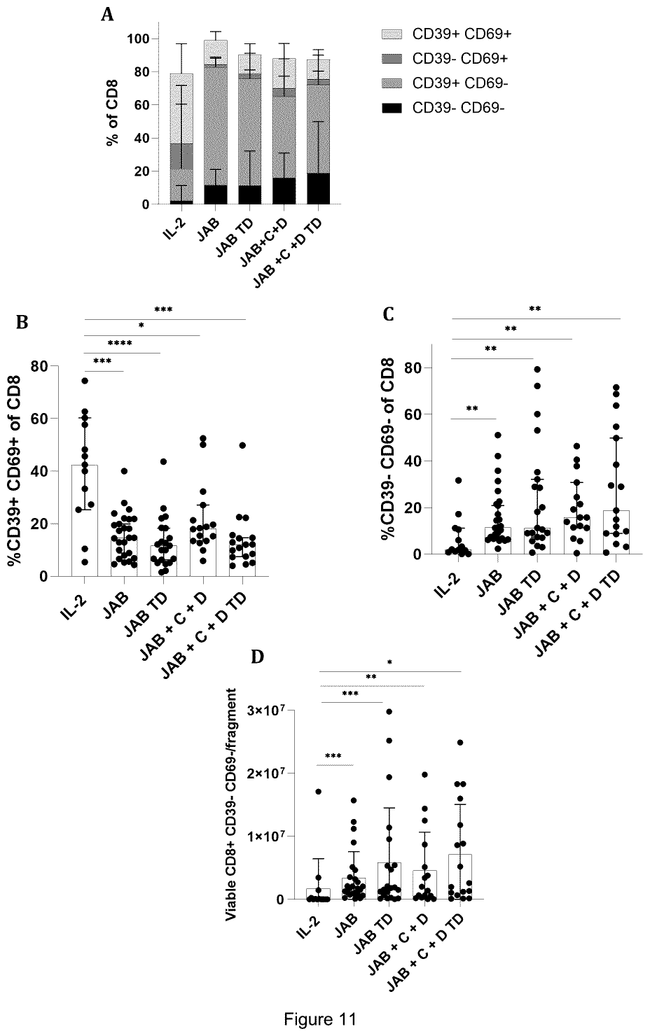

Example 12 illustrated in FIG. 11 demonstrated that adding a combination of TME stimulators as described in Example 6 to the standard young TIL protocol and staining T cells using anti-CD3, anti-CD8, anti-CD39 and anti-CD69 flow cytometry antibodies as described in example 8 results in an increase of the frequency of CD39-CD69− CD8 TILs and a decrease of CD39+ CD69+ CD8+ T cells compared to the standard IL-2 condition, as shown in FIGS. 11A, 11B and 11C. This effect was most pronounced for TILs expanded with JAB stimulators added with a time delay (TD).

This was illustrated using a representative number of tumor fragments from ovarian cancer, cervical cancer, lung cancer and renal cell carcinoma.

CD39-CD69− cells have been shown to be correlated with response to ACT in melanoma patients, as especially higher numbers of double negative cells are significantly higher in patients that respond to therapy. These cells were shown to exhibit a stem-like phenotype characterized by self-renewal capacity to be able to reconstitute the cytotoxic effector cell population upon stimulation (Krishna, S et al., Stem-like CD8 T cells mediate response of adoptive cell immunotherapy against human cancer, Science 370, 1328-1334 (2020).

### Example 13—TME Stimulators Added with a Time Delay of 2 Days Lead to a Product with a Favorable Phenotype

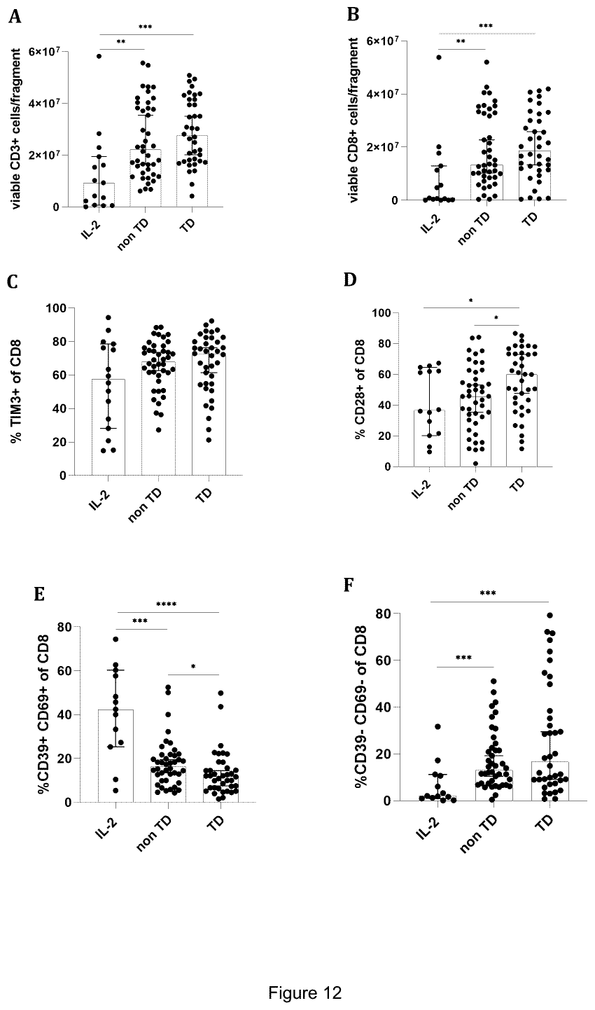

Example 13 illustrated in FIG. 12 demonstrated that adding a combination of TME stimulators as described in Example 6 to the standard young TIL protocol and staining T cells using anti-CD3, anti-CD8, anti-CD39 and anti-CD69, anti-TIM3, anti-CD28 flow cytometry antibodies as described in example 8 and comparing TIL samples expanded with IL-2, and TME stimulators with or without TD, resulted in significantly higher numbers of CD3+ and CD8+ TILs per fragment in samples expanded with TME stimulators added with a time delay of 2 days compared to TME stimulators added without time delay (FIG. 12 A, B). There was a tendency towards higher TIM3 expression as well as a significantly higher proportions of cells expressing the coactivation marker CD28 in TD samples, which points towards cells that are activated and tumor-specific (FIG. 12 C, D). Frequencies of CD39+ CD69+ cells were significantly lower in TD than in non TD samples, whereas the frequency of CD39−CD69− cells had the tendency to be higher in TD samples compared to non TD samples (FIG. 12 E, F).

Summarizing, comparing the phenotype of TILs expanded with TME stimulators of group A and B or A, B, C, D and adding J with a time delay of 2 days resulted in higher numbers of relevant, tumor specific TILs with a favorable phenotype.

### Example 14—Cytotoxic Potential Analysis of “Young” TIL Cultures with TME Stimulators

This example demonstrates the analysis of the cytotoxic potential of “young” TIL cultures with TME stimulators performed as described in example 6.

When cultures designated for young TIL generation were harvested, their reactivity and cytotoxic potential was assessed by flow cytometry. Reactivity was assessed by stimulation of young TILs with αCD3/αCD28/αCD137 coated beads and subsequent staining of cytotoxic degranulation marker CD107a on the cell surface and cytokines INFg and TNFa intracellularly. Characterization of T cell subsets was additionally analyzed using following markers:

TIL cell surface: CD107a, CD3, CD4, CD8, Live-Dead

TIL intracellularly: INFg, TNFa

Briefly, about 2×10⁶ young TILs per sample were thawed and rested overnight in a 24-well plate in RPMI+10% inactivated human AB serum and 1% Pen/Strep. The next day, cells were harvested and counted. 1×10⁵ TILs were transferred to a 96 well plate in triplicates and stimulated with αCD3/αCD28/αCD137 dynabeads with a bead-to-cell ratio of 1:20 for six hours in presence of αCD107a antibody and Golgi Plug.

After six hours, cells were washed and then incubated with titrated surface antibodies (BD Biosciences, Table 1) and PBS for 30 min at 4° C. Cells were washed twice with PBS+0.5% BSA and then fixed overnight at 4° C. with fixation buffer (FoxP3 Staining Buffer Set, ebioscience, Table 1). The next day cells were washed twice with Permeabilization buffer (FoxP3 Staining Buffer Set ebioscience, Table 1) and then stained for intracellular cytokine antibodies (BD biosciences, Table 1) and PBS for 30 min at 4° C. Cells were washed twice with Permeabilization buffer (FoxP3 Staining Buffer Set, ebioscience, Table 1), resuspended in PBS+0.5% BSA and directly analyzed by flow cytometry (CytoFLEX, Beckman Coulter).

This example demonstrates the reactivity and functionality analysis of “young” TIL cultures with TME stimulators.

### Example 15—TME-Stimulators in Combination Added with Time Delay Enhance the Frequency and Total Number of TILs Triple-Positive for Cytotoxic Markers

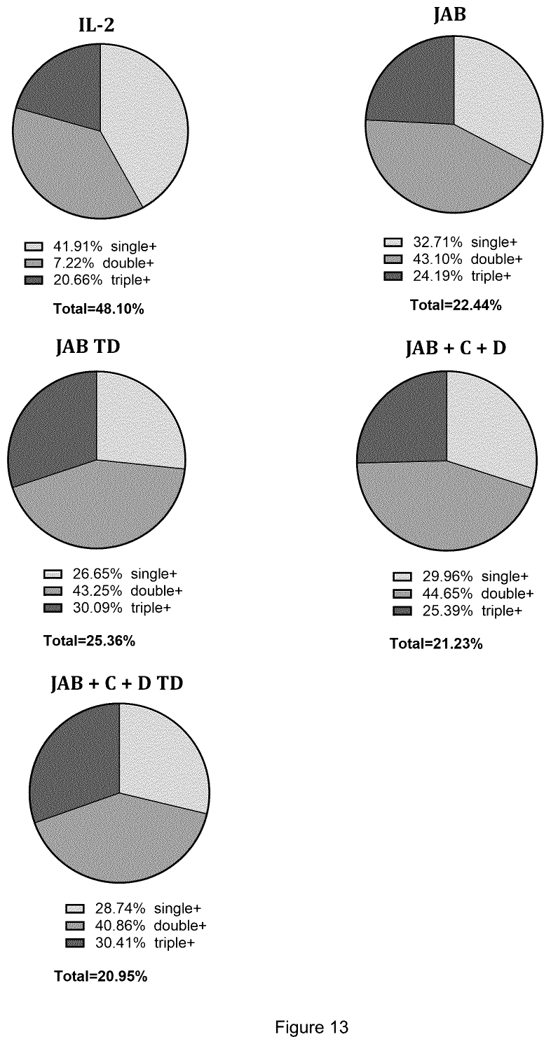

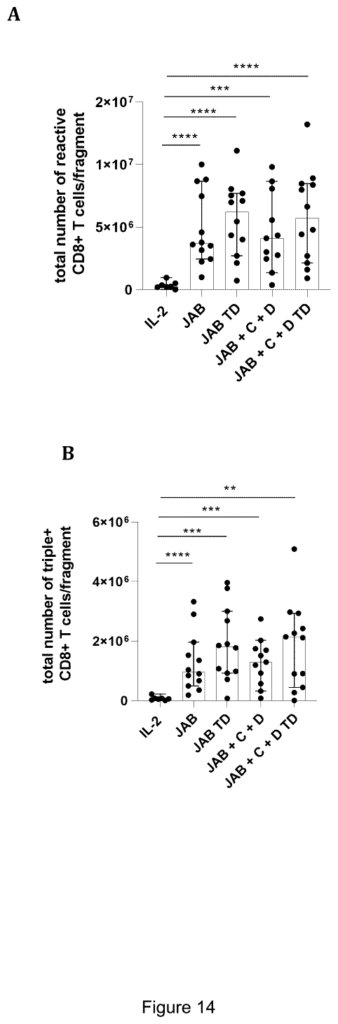

Example 15 illustrated in FIGS. 13, 14 and 15 demonstrated that adding a combination of TME stimulators as described in Example 6 to the standard young TIL protocol with or without a time delay of 2 days and stimulating T cells using αCD3, αCD28 and a41BB coated beads followed by an (intra-)cellular staining with αCD107a, αCD3, αCD4, αCD8, alFNg and aTNFa as described in example 14 resulted in an increased frequency of CD8+ T cells expressing all three of the cytotoxic markers, especially in TILs expanded with JAB TD and JAB+C+D TD (FIG. 13). FIG. 14 A shows, that the total number of reactive cells was significantly increased in all samples expanded with TME stimulators but especially in TD samples. A similar tendency could be seen for the total number of triple-positive TILs, shown in FIG. 14 B.

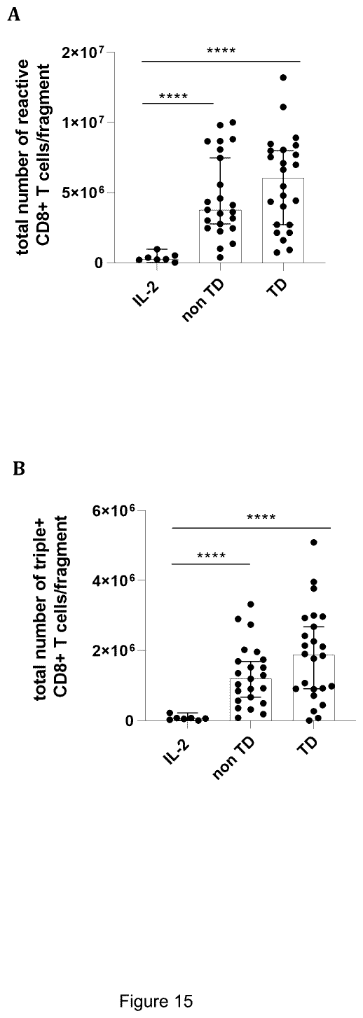

The effect of the time delay is again illustrated in FIG. 15 with a direct comparison between TME stimulators added with or without time delay. There was a clear tendency towards a higher number of total reactive CD8+ T cells and a higher number of triple positive CD8+ T cells per tumor fragment, when stimulators are added with a time delay of 2 days.

Summarized, TILs expanded with TME stimulators added with a 2-day time delay showed a higher frequency and total number of cells that are activated upon bead stimulation and specifically higher number of cells expressing all three markers IFNg, TNFa and CD107a, shown to exhibit a higher capacity for antigen recognition and cytotoxicity.

This was illustrated using a representative number of tumor fragments from ovarian cancer and renal cell carcinoma.

### Example 16—TME Stimulators in Combination Added with Time Delay Clearly Enhances the Frequency of Reactive Cells in Selected Patient Samples

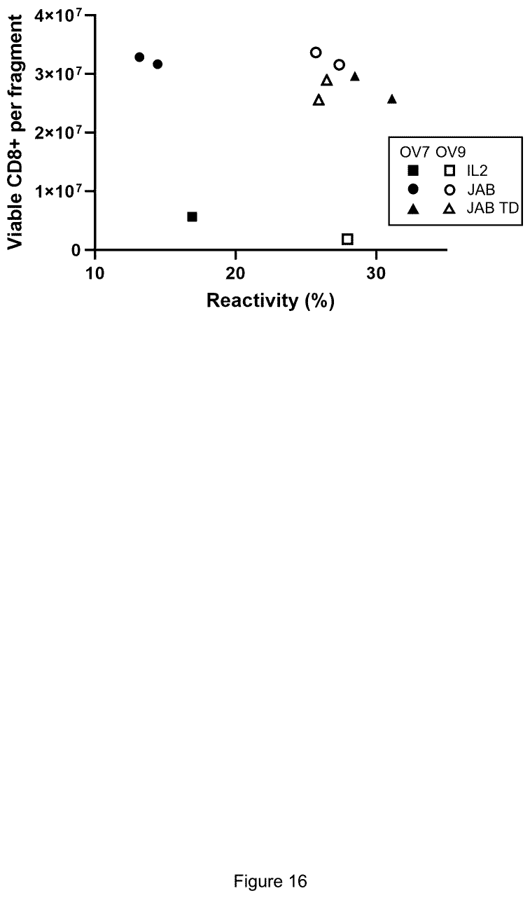

Example 16 illustrated in FIG. 16 demonstrated that adding a combination of TME stimulators as described in Example 6 to the standard young TIL protocol with or without a time delay of 2 days and stimulating T cells using αCD3, αCD28 and a41BB coated beads followed by an (intra-)cellular staining with αCD107a, αCD3, αCD4, αCD8, alFNg and aTNFa as described in example 14, resulted in a higher frequency of reactive cells in ovarian cancer patient OV7 in TILs expanded with JAB TD compared to TILs expanded with JAB without time delay. This difference was not associated with a decrease in expansion. The difference in reactivity was not seen for OV9, where reactivity was similarly high in JAB and JAB TD samples.

In summary, this example shows, that adding TME stimulators with a time delay of 2 days can result in a higher reactivity after unspecific bead stimulation for some patients, whereas it does not make a difference in other patients, Therefore, adding TME stimulators with a time delay seems to be advantageous to increase cytotoxic potential of the TIL product while retaining proliferation of TILs.

### Example 17—Analysis of CD8+ T Cells Specificities with a Panel of 30 Cancer-Associated pMHC Tetramers

This example demonstrates the analysis of the T cell specificities within the different TIL products expanded with and without TME stimulators as described in Example 6 to the standard young TIL protocol with or without a time delay of 2 days.

This was illustrated using a representative number of tumor fragments from three cervical cancer patients that are positive for the HLA allele A0201. Of two patients, IL-2 samples were available. All samples were expanded from five tumor fragments unless otherwise indicated.

Tetramers represent a selection of 30 peptides bound to an HLA 0201 molecule, the majority of peptides derived from Cancer-Testis proteins well known to be expressed in numerous tumor entities and recognized by T cells (Table 5). Other peptides are derived from proteins found to be overexpressed in some cancer entities while a small fraction is derived from melanocytic peptides that play a role in melanoma progression and are therefore not relevant in cervical cancer.

When cultures designated for young TIL generation were harvested, their specificities were assessed by staining with a panel of 30 different pMHC tetramers color-coded with unique combinations of two fluorophores each, and subsequently analyzed by flow cytometry. Reactivity was defined by >0.001%, >10 specific CD8+ cells and by inspecting tetramer+ populations.

### Characterization of T Cell Subsets was Additionally Analyzed Using Following Markers:

### CD3, CD8, Live-Dead

In short, HLA A0201 monomers were incubated with the library of 30 cancer-associated peptides to load peptides onto the HLA molecules. Subsequently, pMHC monomers were labeled with two different streptavidin-fluorophores in separate wells with unique combinations for each individual pMHC combination (Table 4). After incubation, the combi-coded pMHC tetramer library was mixed and TIL samples were stained with the tetramer library followed by a surface antibody staining with anti-CD3, anti-CD8 and Live-Dead. Antigen-specific cells were identified by gating on double positive cells for each relevant combination AND negative for other fluorophores.

### Example 18—Cancer Antigen Specific CD8+ T Cells are Present in Cervical Cancer Patients with a Higher Number and Frequency in TILs Expanded with JAB TD

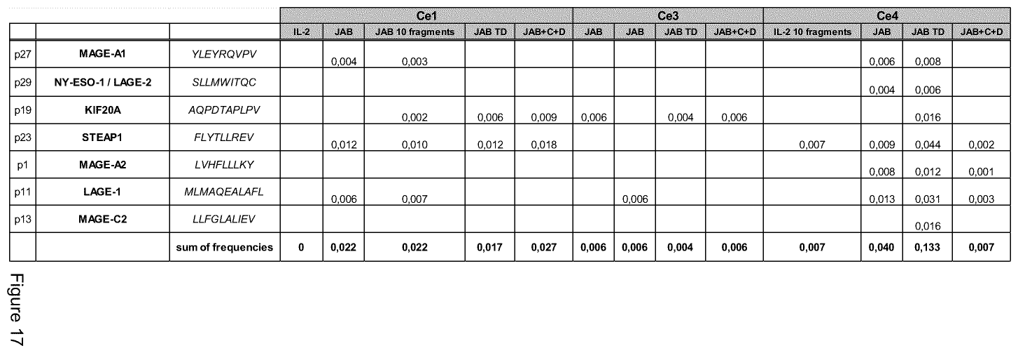

Example 18 illustrated in FIG. 17 demonstrated that by adding a combination of TME stimulators as described in Example 6 to the standard young TIL protocol with or without a time delay of 2 days and staining with a tetramer library as described in example 17, T cell populations specific for cancer-testis and overexpressed antigens can be identified in TIL samples expanded from cervical cancer patients. FIG. 17 shows seven different peptides recognized by CD8+ T cells across the three selected cervical cancer patients and the corresponding TIL samples grown with TME stimulators with or without time delay or the IL-2 condition. The number represents the frequency of the specific population and the number in the bottom the sum thereof.

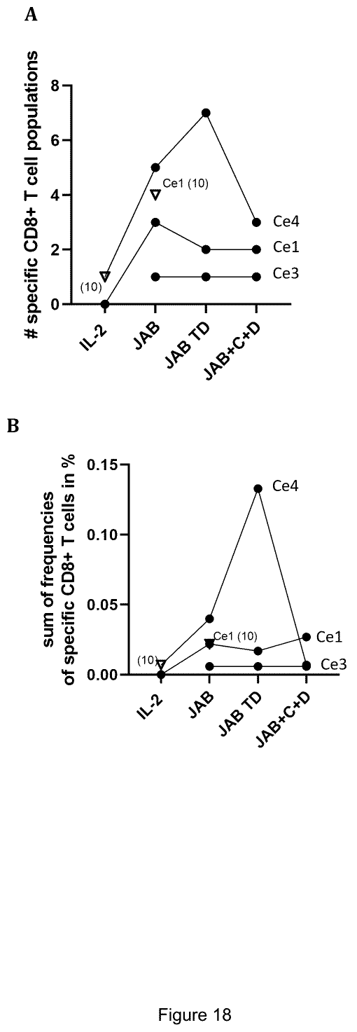

FIG. 18 A illustrates the total number of specific CD8+ T cell populations and FIG. 18 B the sum of frequencies of all present populations per patient. TILs expanded with IL-2 showed one (Ce4) or no (Ce1) specific CD8+ T cell population. In contrast, TILs expanded with JAB showed five and three populations, respectively. The JAB TD sample from Ce4 showed an increase to seven populations. Ce3 showed the same number of populations across all available samples, an IL-2 sample was not available due to lack of cells to analyze.

JAB+C+D samples exhibited some T cell populations but in general to a lower number and frequency compared to JAB and JAB TD. JAB+C+D TD samples were not available for these patients.

Of note, most (five) peptides derive from cancer/testis antigens, while STEAP1 and KIF20A represent overexpressed antigens, of which KIF20A has been described to be overexpressed in cervical cancer (Zhang, W et al., High Expression of KIF20A Is Associated with Poor Overall Survival and Tumor Progression in Early-Stage Cervical Squamous Cell Carcinoma, PLoS 11(12):e0167449 (2016))

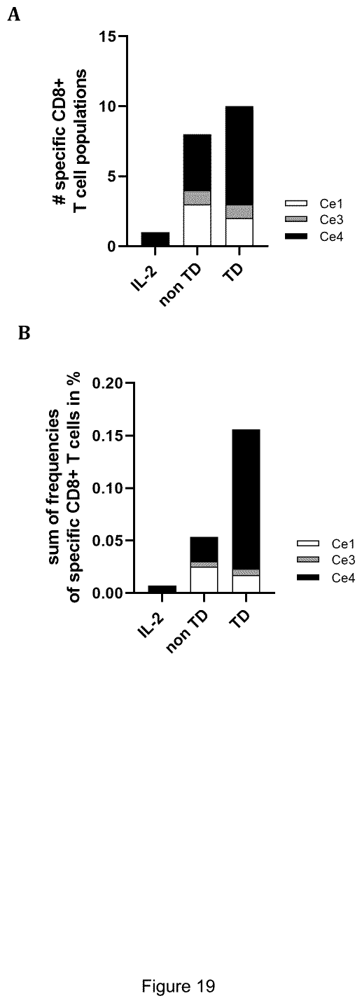

FIG. 19 summarizes the number and frequency of specific CD8 T cells populations of non TD and TD samples, compared to IL-2 samples, illustrating higher total numbers and frequencies of cancer-testis and overexpressed antigen specific CD8 T cells in both non TD and TD samples, with the tendency towards higher numbers and specifically higher frequencies in TD samples.

It has previously been shown that responders of adoptive cell therapy showed a significant higher number and frequency of neo-antigen specific T cells and that a high number correlated with better survival. It therefore seems to be crucial to broaden the T cell repertoire in the TIL product (Heeke, C. et al., Neoantigen-reactive CD8+ T cells affect clinical outcome of adoptive cell therapy with tumor-infiltrating lymphocytes in melanoma. J Clin Invest; 132(2)). This data showed that cancer specific CD8+ T cells can be detected in cervical cancer patients and by adding TME stimulators, especially with a time delay of 2 days, the number and frequency of these populations can be increased, therefore broadening the T cell repertoire and potentially making adoptive cell therapy more successful.

### Example 19—“Young” Tumor-Infiltrating Lymphocytes (TILs) with TME Stimulators

This example demonstrated the generation of “young” tumor-infiltrating lymphocytes (TILs) with TME stimulators as described in Example 6 with following changes:

The fresh or frozen tumors were divided into 1-3 mm³ fragments and placed into a G-Rex 6-well plate (WilsonWolf; 5 fragments per well) with 5 ml complete medium (CM) supplemented with 6000 IU/mL IL-2 (Clinigen) only (baseline) or in combination with TME stimulators of each of the PD-1/PD-L1 antagonists (group A), CTLA-4 antagonist (group B), and 4-1 BB agonist (group J) in combination with anti-CD3, in a humidified 37° C. incubator with 5% CO₂ at the same time or with a time delay or time lapse of 48 h or 96 h. TME stimulation combinations are called corresponding to the stimulator groups J, A, B, without or with time delay (TD) and relevant time delay in hours.

### Example 20—Culturing TILs with TME Stimulators Increases Cell Number and Success Rate while Reducing Culture Time

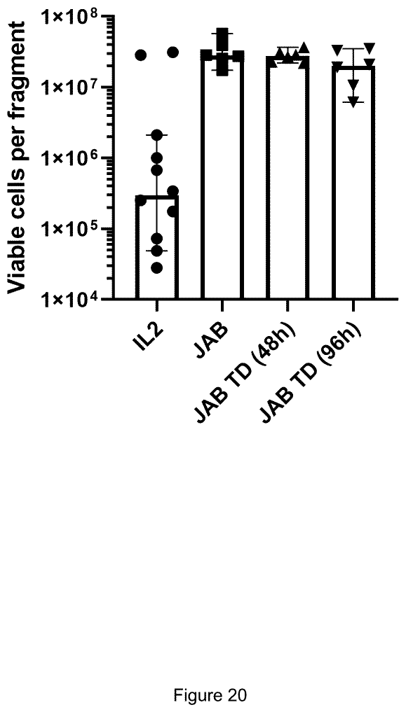

Example 20 illustrated in FIG. 20 demonstrated that adding TME stimulators as described in Example 19 to the standard young TIL protocol leads to similar numbers of viable cells per fragment. Surprisingly, adding J with a TD of 96 hours leads to similar number of cells compared to the 48 h TD, although slightly lower compared to the 48 h time delay or no time delay. This could be due to the fact that TILs were expanded from frozen fragments, whereas other conditions derived from fresh tumor fragments.

This was illustrated using a representative number of tumor fragments from ovarian cancer, renal cell carcinoma and cervical cancer.

### Example 21—Culturing TILs with TME Stimulators and an Increased Time Delay does not Change the Composition of the TIL Product

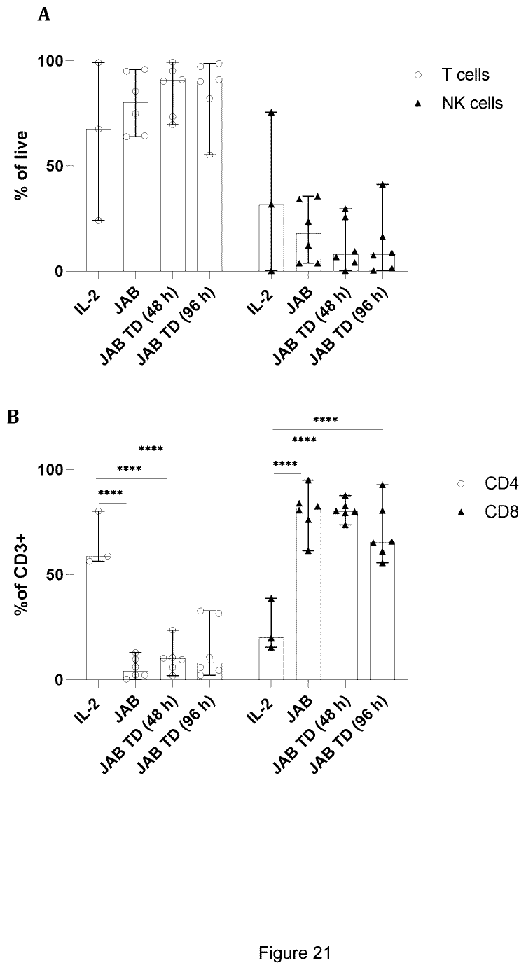

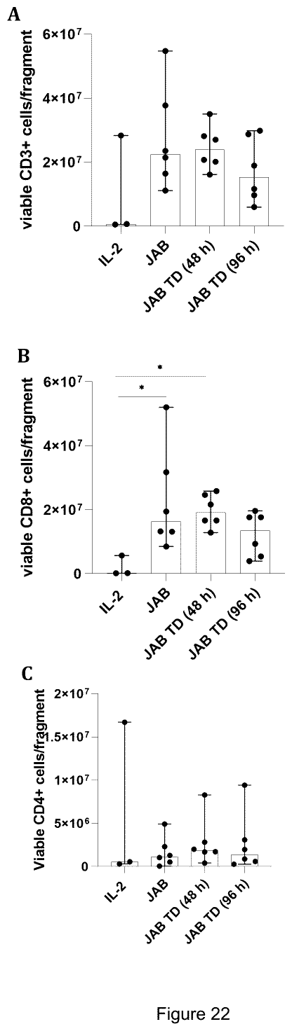

Example 21 illustrated in FIGS. 21 and 22 demonstrated that adding TME stimulators as described in Example 19 to the standard young TIL protocol with or without a time delay of 48 or 96 hours and stained with anti-CD3, anti-CD4, anti-CD56 and anti-CD8 antibodies as described in Example 8 led to similar or slightly higher frequencies of CD3+ cells and decreased frequencies of NK cells across all samples with TME stimulators compared to the IL-2 condition, with no differences between the 96 h and 48 h time delay (FIG. 21 A). The frequencies of CD4+ T cells were decreased and CD8 frequencies increased compared to the IL-2 condition with again only slight differences between the different TME stimulator conditions (FIG. 21 B).

FIG. 22 A illustrates that total numbers of CD3+ T cells in samples expanded with TME stimulators were consistently higher than in the IL-2 condition, although not significant. Numbers of total CD8+ T cells tend to be slightly lower in samples receiving JAB with a 96 h time delay compared to JAB or JAB TD 48 h whereas numbers of CD4+ T cells do not show any difference across all samples (FIG. 22 B, C)

This was illustrated using a representative number of tumor fragments from ovarian cancer, renal cell carcinoma and cervical cancer.

In total, this data showed that adding TME stimulators with a time delay of 96 h leads to a high cell expansion without compromising on CD8+ T cell numbers.

### Example 22—Culturing TILs with TME Stimulators and an Increased Time Delay Leads to an Increased Expression of Activation Markers BTLA, LAG3 and TIM3 while CD28 Expression is Retained

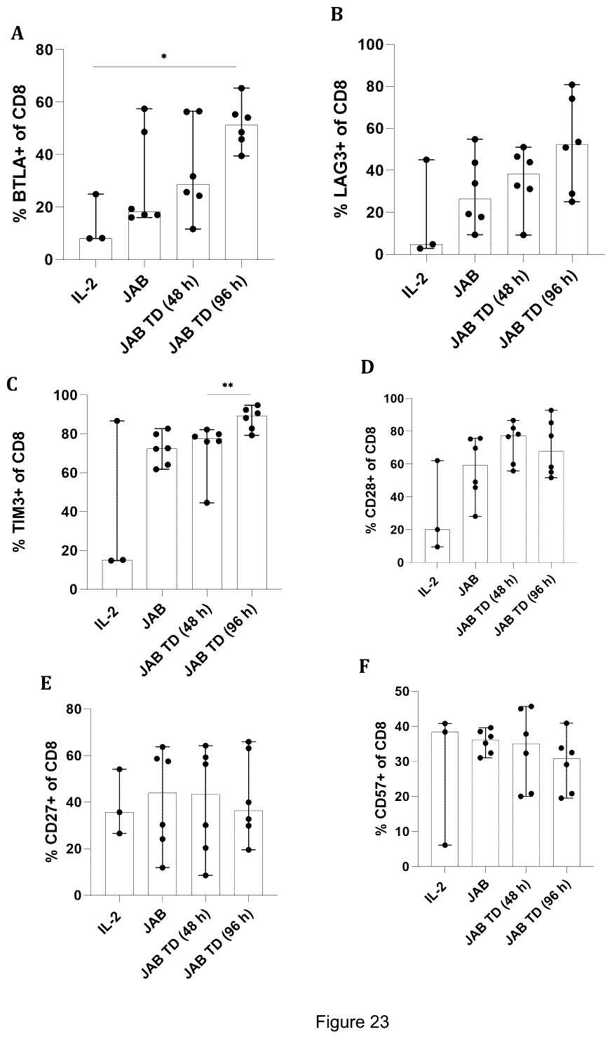

Example 22 illustrated in FIG. 23 demonstrated that adding TME stimulators as described in Example 19 to the standard young TIL protocol with or without a time delay of 48 or 96 hours and stained with anti-CD3, anti-CD8, anti-BTLA, anti-LAG3, anti-TIM3, anti-CD28, anti-CD27 and anti-CD57 antibodies as described in Example 8 led to an increased expression of activation markers BTLA, LAG3 and TIM3 in TIL samples expanded with TME stimulators with most significant differences in samples expanded with a 96 h time delay (FIG. 23 A, B, C). Expression of CD28 is elevated in all TIL samples expanded with TME stimulators but seemed to be slightly higher in TILs expanded with the 96 h time delay (FIG. 23 D).

At the same time, expression of CD27 and CD57 remained unchanged in the JAB TD 96 h samples compared to JAB or JAB TD (FIG. 23 E, F).

This was illustrated using a representative number of tumor fragments from ovarian cancer, renal cell carcinoma and cervical cancer.

These differences point toward the expansion of more tumor specific and activated cells, that retain the expression of costimulatory molecules like CD28 and therefore beneficial for tumor recognition.

### Example 23—Culturing TILs with TME Stimulators and an Increased Time Delay Leads to an Increased Population of Terminally Differentiated Cells (Temra)

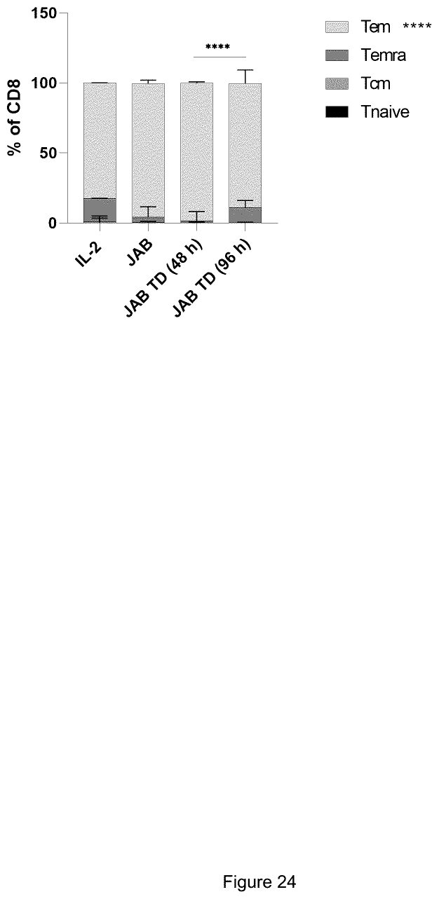

Example 23 illustrated in FIG. 24 demonstrated that adding TME stimulators as described in Example 19 to the standard young TIL protocol with or without a time delay of 48 or 96 hours and stained with anti-CD3, anti-CD8, anti-CCR7 and antiCD45RA antibodies as described in Example 8 led to a significantly bigger population of CCR7− CD45RA+ CD8+ Temra cells in the TIL samples expanded with JAB and a 96 h time delay compared to samples without or 48 h time delay.

This was illustrated using a representative number of tumor fragments from ovarian cancer, renal cell carcinoma and cervical cancer.

Terminally differentiated Temra cells are usually more cytotoxic but might be more exhausted and not as proliferative as effector-memory cells, that are predominantly present in the other TME stimulator samples. Adding the JAB TME stimulators at day 0 reduces the frequency of Temra cells compared to IL-2 alone. The time delay of 48 hours seems to further reduce this Temra population, whereas the 96 hour time delay reverts this trend mimicking the IL-2 alone data.

### Example 24—Culturing TILs with TME Stimulators and an Increased Time Delay Leads to an Expansion of CD8+ T Cells that are Negative for CD39 and CD69 with a Stem-Cell Like Phenotype

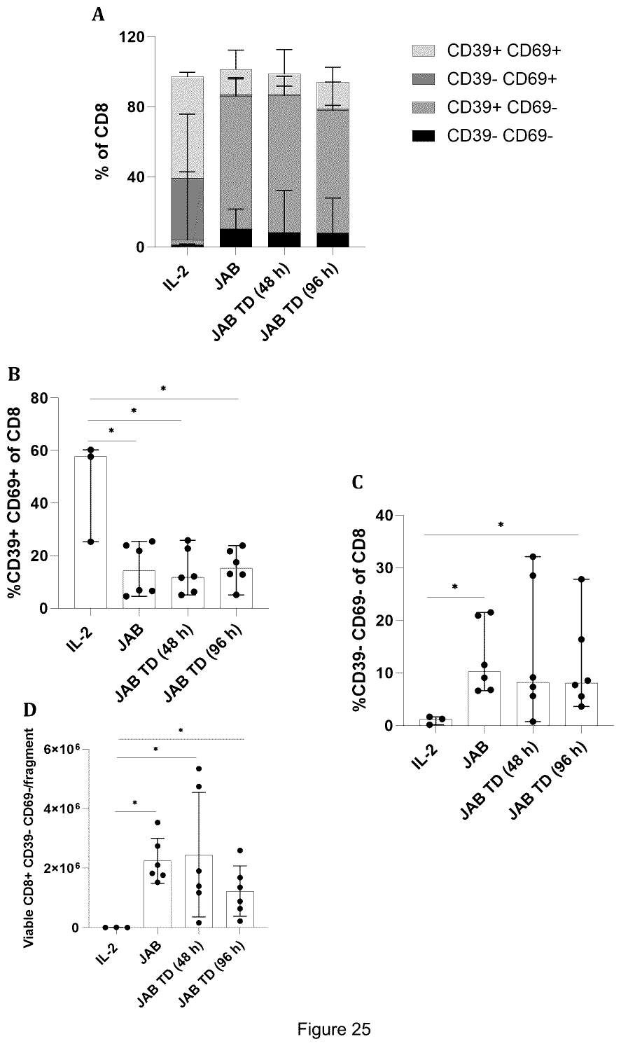

Example 24 illustrated in FIG. 25 demonstrated that adding TME stimulators as described in Example 19 to the standard young TIL protocol with or without a time delay of 48 or 96 hours and stained with anti-CD3, anti-CD8, anti-CD39 and anti-CD69 antibodies led to a decrease in the CD39+ CD69+ CD8+ T cell population in samples expanded with TME stimulators and an increase in the double negative fraction compared to the IL-2 samples. There were no significant differences between JAB, JAB TD 48 h and 96 h. Also the total numbers of CD39-CD69− cells were mostly similar although the 96 h time delay seemed to lead to slightly lower total numbers of this population which might be due to a slightly lower expansion in total.

Summarized, this data shows that as discussed in Example 12, adding TME stimulators to the TIL cultures led to an increased expansion of CD39-CD69− cells that have been described to have a stem cell like phenotype, that seemed to be clinically relevant in ACT trials.

Summarizing Examples 20-24, expanding TILs with TME stimulators with a time delay of 96 h led to a similar expansion of desired CD8+ T cells compared to the shorter 48 h time delay. These CD8+ T cells show a favorable activated and potentially tumor specific phenotype.

### Items

1. Expanded tumor infiltrating lymphocytes (TILs) for use in treating a subject with cancer, the treatment comprising the steps of:
a) culturing autologous T cells by obtaining a first population of TILs from a tumor resected from a mammal,
b) performing a depletion of suppressive cells, including regulatory T cells, and/or blocking negative signals by the addition of one or more TME stimulators from the group of “Inhibitors” to obtain a depleted population of TILs,
c) performing a first expansion by culturing the depleted population of TILs in a cell culture medium comprising:
   one or more TME stimulators from the group of “cytokines”, and/or
   one or more of the TME stimulators from the “Stimulator” group to produce a second population of TILs,
d) performing a second expansion by supplementing the cell culture medium of the second population of TILs with additional IL-2 and/or other cytokines from the “cytokine” group, anti-CD3 antibody, and antigen presenting cells (APCs), to produce a third population of TILs, wherein the third population of TILs is a therapeutic population; and
e) after administering nonmyeloablative lymphodepleting chemotherapy, administering to the mammal the therapeutic population of T cells, wherein the T cells administered to the mammal, whereupon the regression of the cancer in the mammal is promoted.

2. Expanded tumor infiltrating lymphocytes (TILs) for use in promoting regression of a cancer in a subject with cancer, the regression comprising the steps of:
a) culturing autologous T cells by obtaining a first population of TILs from a tumor resected from a mammal,
b) performing a depletion of suppressive cells, including regulatory T cells, and/or blocking negative signals by the addition of one or more TME stimulators from the group of “Inhibitors” to obtain a depleted population of TILs,
c) performing a first expansion by culturing the depleted population of TILs in a cell culture medium comprising:
   one or more TME stimulators from the group of “cytokines”, and/or
   one or more of the TME stimulators from the “Stimulator” group to produce a second population of TILs,
d) performing a second expansion by supplementing the cell culture medium of the second population of TILs with additional IL-2, anti-CD3 antibody, and antigen presenting cells (APCs), to produce a third population of TILs, wherein the third population of TILs is a therapeutic population; and
e) after administering nonmyeloablative lymphodepleting chemotherapy, administering to the mammal the therapeutic population of T cells, wherein the T cells administered to the mammal, whereupon the regression of the cancer in the mammal is promoted.

3. A method for expanding tumor infiltrating lymphocytes (TILs) into a therapeutic population of TILs comprising:
a) culturing autologous T cells by obtaining a first population of TILs from a tumor resected from a mammal,
b) performing a depletion of suppressive cells, including regulatory T cells, and/or blocking negative signals by the addition of one or more TME stimulators from the group of “Inhibitors” to obtain a depleted population of TILs,
c) performing a first expansion by culturing the depleted population of TILs in a cell culture medium comprising:
   one or more TME stimulators from the group of “cytokines”, and/or
   one or more of the TME stimulators from the “Stimulator” group to produce a second population of TILs,
d) performing a second expansion by supplementing the cell culture medium of the second population of TILs with additional IL-2, anti-CD3 antibody, and antigen presenting cells (APCs), to produce a third population of TILs, wherein the third population of TILs is a therapeutic population.

4. The uses and methods of items 1-3, wherein one or more TME stimulators from the group of “cytokines” are added in step b).

5. The uses and methods of items 1-4, wherein the group of “cytokines” are selected from the group consisting of IL-2, IL-7, IL-12, IL-15, and IL-21.

6. The uses and methods of items 1-5, wherein the group of “Inhibitors” are selected from the group consisting one or more of:
A) substances that act through the PD-1 receptor on T-cells,
B) substances that act through the CTLA-4 receptor on T-cells,
C) substances that act through the LAG-3 receptor on T-cells,
D) substances that act through the TIGIT/CD226 receptor on T-cells.
E) substances that act through the KIR receptor on T-cells,
F) substances that act through the TIM-3 receptor on T-cells,
G) substances that act through the BTLA receptor on T-cells, and
H) substances that act through the A2aR receptor on T-cells.

7. The uses and methods of items 1-5, wherein the group of “Inhibitors” are selected from the group consisting one or more of:
A) substances that act through the PD-1 receptor on T-cells,
B) substances that act through the CTLA-4 receptor on T-cells,
C) substances that act through the LAG-3 receptor on T-cells, and
D) substances that act through the TIGIT/CD226 receptor on T-cells.

8. The uses and methods of items 1-7, wherein the substance of group A is selected from one or more from the group consisting of pembrolizumab, nivolumab, cemiplimab, sym021, atezolizumab, avelumab, and durvalumab.

9. The uses and methods of items 1-8, wherein the substance of group B is selected from one or more from the group consisting of ipilimumab and tremelimumab.

10. The uses and methods of items 1-9, wherein the substance of group C is selected from one or more from the group consisting of relatlimab, eftilagimo alpha, and sym022.

11. The uses and methods of items 1-10, wherein the substance of group D is tiragolumab.

12. The uses and methods of items 1-11, wherein the group of “Inhibitors” are:
A: substances that act through the PD-1 receptor on T-cells, and
B: substances that act through the CTLA-4 receptor on T-cells.

13. The uses and methods of items 1-12, wherein the group of “Inhibitors” are:
A: substances that act through the PD-1 receptor on T-cells,
B: substances that act through the CTLA-4 receptor on T-cells, and
C) substances that act through the LAG-3 receptor on T-cells.

14. The uses and methods of items 1-13, wherein the group of “Inhibitors” are:
A: substances that act through the PD-1 receptor on T-cells,
B: substances that act through the CTLA-4 receptor on T-cells, and
D) substances that act through the TIGIT/CD226 receptor on T-cells.

15. The uses and methods of items 1-14, wherein the group of “Inhibitors” are:
A) substances that act through the PD-1 receptor on T-cells,
B) substances that act through the CTLA-4 receptor on T-cells,
C) substances that act through the LAG-3 receptor on T-cells, and
D) substances that act through the TIGIT/CD226 receptor on T-cells.

16. The uses and methods of items 1-15, wherein the group of “Inhibitors” are selected from the group consisting one or more of:
P) epacadostat,
Q) substances that act through the TGFβ receptor on T-cells,
R) substances that act through the IL-10 receptor on T-cells, and
S) substances that act through the IL-35 receptor on T-cells.

17. The uses and methods of items 1-16, wherein the group of “Inhibitors” are selected from the group consisting one or more of:
T) cyclophosphamides,
U) TKIs,
V) substances that act through αCD25, and
X) IL2/Diphteria toxin fusions.

18. The uses and methods of items 1-17, wherein the group of “Stimulator” are selected from the group consisting one or more of:
I) substances that act through the OX40/CD134 receptor on T-cells,
J) substances that act through the 4-1BB/CD137 receptor on T-cells,
K) substances that act through the CD28 receptor on T-cells,
L) substances that act through the ICOS receptor on T-cells,
M) substances that act through the GITR receptor on T-cells,
N) substances that act through the CD40L receptor on T-cells,
O) substances that act through the CD27 receptor on T-cells, and
W) substances that act through CD3 on T cells.

19. The uses and methods of item 18, wherein the group of “Stimulator” is:
J) substances that act through the 4-1BB/CD137 receptor on T-cells.

20. The uses and methods of item 11, wherein the substance of group J is selected from one or more from the group consisting of urelumab and utomilumab.

21. The uses and methods of items 1-20, wherein:
the group of “Inhibitors” in step b) are:
   A: substances that act through the PD-1 receptor on T-cells, and
   B: substances that act through the CTLA-4 receptor on T-cells, and
wherein the group of “Stimulator” in step c) is:
   J) substances that act through the 4-1BB/CD137 receptor on T-cells.

22. The uses and methods of items 1-21, wherein step b) and step c) are performed in time lapse, i.e. one day apart, or such as 2, 3, 4, 5, 6 or 7 days apart.

23. The uses and methods of item 22, wherein the step step b) and step c) are performed 1-2 days apart.

24. The uses and methods of item 22, wherein the step step b) and step c) are performed 1-3 days apart.

25. The uses and methods of item 22, wherein the step b) and step c) are performed 1-4 days apart.

26. The uses and methods of item 22, wherein the step b) and step c) are performed 1-5 days apart.

27. The uses and methods of item 22, wherein the step b) and step c) are performed 1-6 days apart.

28. The uses and methods of item 22, wherein the step b) and step c) are performed 1-7 days apart.

29. The uses and methods of item 22, wherein the step b) and step c) are performed 2-4 days apart.

30. The uses and methods of item 22, wherein the step b) and step c) are performed 4-8 days apart.

31. The uses and methods of items 1-30, wherein the concentration of the substance is 0.1 μg/mL to 300 μg/mL, such as 1 μg/mL to 100 μg/mL, such as 10 μg/mL to 100 μg/mL, such as 1 μg/mL to 10 μg/mL, such as 2-20 μg/mL.

32. The uses and methods of items 1-31, wherein steps (a) through (b) are performed within a period of about 7 days to about 28 days.

33. The uses and methods of items 1-32, wherein step (c) is performed within a period of about 7 days to about 21 days.

34. The uses and methods of items 1-33, wherein the therapeutic population of T cells is used to treat a cancer type selected from the groups consisting of breast cancer, renal cell cancer, bladder cancer, melanoma, cervical cancer, gastric cancer, colorectal cancer, lung cancer, head and neck cancer, ovarian cancer, Hodgkin lymphoma, pancreatic cancer, liver cancer, and sarcomas.

35. The uses and methods of items 1-34, wherein step (c) results in 1×10⁷ to 1×10¹² cells, such as 1×10⁸ to 5×10⁹ cells, such as 1×10⁹ to 5×10⁹ cells, such as 1×10⁸ to 5×10¹⁰ cells, such as 1×10⁹ to 5×10¹¹ cells.

36. The uses and methods of items 1-35, wherein the anti-CD3 antibody is OKT3.

37. The uses and methods of items 1-36, wherein the mammal is a human individual.

38. The uses and methods of items 1-37, wherein the antibody is selected from the group consisting of a monoclonal antibody, a human antibody, a humanized antibody, a chimeric antibody, a murine antibody, a F(ab′)2 or Fab fragment, and a Nanobody.

39. The uses and methods of items 1-38, wherein group A is selected from one or more from the group consisting of pembrolizumab, nivolumab, cemiplimab, sym021, atezolizumab, avelumab, durvalumab, Toripalimab, Sintilimab, Camrelizumab, Tislelizumab, Sasanlimab, Dostarlimab, MAX-10181, YPD-29B, IMMH-010, INCB086550, GS-4224, DPPA-1, TPP-1, BMS-202, CA-170, JQ1, eFT508, Osimertinib, PlatycodinD, PD-LYLSO, Curcumin, and Metformin.

40. The uses and methods of items 1-39, wherein group B is selected from one or more antibodies from the group consisting of ipilimumab and tremelimumab.

41. The uses and methods of items 1-39, wherein the substance of group J is selected from one or more from the group consisting of urelumab, utomilumab, BCY7835, and BCY7838.

42. A population of tumor infiltrating lymphocytes (TILs) obtainable by a method of any of the previous items.

43. A population of tumor infiltrating lymphocytes (TILs) comprising a clinically relevant number of TILs with a higher percentage of CD8 T cells expressing markers associated with tumor-specificity (exhaustion markers).

1. A method for expanding tumor infiltrating lymphocytes (TILs) into a therapeutic population of TILs comprising:
a) culturing autologous T cells by obtaining a first population of TILs from a tumor resected from a mammal,
b) performing a depletion of suppressive cells, including regulatory T cells, and/or blocking negative signals in a cell culture medium comprising IL-2 by the addition of one or more TME stimulators from the groups of:
Group A: substances that act through the PD-1 receptor on T-cells, and/or
Group B: substances that act through the CTLA-4 receptor on T-cells,
c) performing a first expansion by culturing the depleted population of TILs in a cell culture medium comprising IL-2, and:
one or more of the TME stimulators from:
Group J: substances that act through the 4-1BB/CD137 receptor on T-cells, to produce a second population of TILs, and
d) performing a second expansion by supplementing the cell culture medium of the second population of TILs with additional IL-2, anti-CD3 antibody, and antigen presenting cells (APCs), to produce a third population of TILs, wherein the third population of TILs is a therapeutic population of TILs.

2-17. (canceled)

18. The method according to claim 1, where step c) further comprises culturing the depleted population of TILs with a substance from Group W: substances that act through the CD3 receptor on T cells.

19. A population of tumor infiltrating lymphocytes (TILs) obtainable by a method of claim 1, wherein the population of TILs has a higher number and frequency of cancer antigen specific T-cell populations than obtainable without adding TME stimulators except IL-2.

20. A method of inhibiting, treating, or promoting the regression of a cancer in a mammal comprising:
administering to the mammal the therapeutic population of TILs obtained by the method of claim 1, wherein the mammal has received a nonmyeloablative lymphodepleting chemotherapy.

21. The method of claim 1, wherein the substances of Group A, Group B and/or Group J are antibodies.

22. The method of claim 21, wherein the antibody is selected from the group consisting of a monoclonal antibody, a human antibody, a humanized antibody, a chimeric antibody, a murine antibody, a F(ab′)2 or Fab fragment, and a Nanobody.

23. The method of claim 1, wherein step b) and step c) are performed 1-4 days apart.

24. The method of claim 20, wherein the cancer is selected from the group consisting of breast cancer, renal cell cancer, bladder cancer, melanoma, cervical cancer, gastric cancer, colorectal cancer, lung cancer, head and neck cancer, ovarian cancer, Hodgkin lymphoma, pancreatic cancer, liver cancer, and sarcomas.

25. The method of claim 1, wherein step (c) results in 1×10⁷ to 1×10¹² cells.

26. The method of claim 1, wherein the anti-CD3 antibody is OKT3.

27. The method of claim 1, wherein the mammal is a human individual.

28. The method of claim 1, wherein group A is selected from pembrolizumab, nivolumab, cemiplimab, sym021, atezolizumab, avelumab, durvalumab, Toripalimab, Sintilimab, Camrelizumab, Tislelizumab, Sasanlimab, Dostarlimab, MAX-10181, YPD-29B, IMMH-010, INCB086550, GS-4224, DPPA-1, TPP-1, BMS-202, CA-170, JQ1, eFT508, Osimertinib, PlatycodinD, PD-LYLSO, Curcumin, or Metformin.

29. The method of claim 1, wherein group B is selected from ipilimumab or tremelimumab.

30. The method of claim 1, wherein the substance of group J is selected from urelumab, utomilumab, BCY7835, or BCY7838.

31. A population of tumor infiltrating lymphocytes (TILs) obtainable by the method of claim 1.

32. A population of tumor infiltrating lymphocytes (TILs) comprising 1×10⁷ to 1×10¹² cells, wherein the population of TILs has a higher percentage of CD8 T cells expressing markers associated with tumour-specificity.
